(19) United States
(12) Patent Application Publication
(10) Pub. No.: US 2007/0049591 A1
(43) Pub. Date: Mar. 1, 2007

(54) Inhibitors of MAPK/Erk Kinase

(75) Inventors: Anthony B. Pinkerton, San Diego, CA (US); Robert L. Davis, Carlsbad, CA (US); Timothy C. Gahman, Encinitas, CA (US); J. Edward Semple, San Diego, CA (US)

Correspondence Address: INTERNATIONAL PATENT GROUP;ATTN: MS LAVERN HALL, P.O. BOX 38129, ST. LOUIS, MO, 63138, US

(73) Assignee: KALYPSYS, INC.

(21) Appl. No.: 11/509,508

(22) Filed: Aug. 24, 2006

Related U.S. Application Data
(60) Provisional application No. 60/711,190, filed on Aug. 25, 2005.

(51) Int. Cl. A61K 31/5377; A61K 31/522; A61K 31/52; A61K 31/519; A61K 31/503; C07D 487/02; C07D 473/02

(52) U.S. Cl. 514/234.5; 514/248; 514/262.1; 514/263.3; 514/263.4; 514/267; 544/235; 544/276; 544/277; 544/250; 544/262

(57) ABSTRACT

The present invention relates to compounds useful as inhibitors of MEK kinase and methods for the treatment or prevention of cellular proliferative disease states, such as conditions related to the hyperactivity of MEK, as well as diseases modulated by the MEK cascade.

This application claims the benefit of priority of U.S. provisional application No. 60/711,190, filed Aug. 25, 2005, the disclosure of which is hereby incorporated by reference as if written herein in its entirety.

### FIELD OF THE INVENTION

The present invention is directed to new compounds, compositions and their application as a pharmaceutical for the treatment of disease. Methods of inhibition of MAPK/Erk Kinase (“MEK” or “Mek”) activity in a human or animal subject are also provided for the treatment of diseases such as cancer, non-cancer hyperproliferative disorders, vascular restenosis, psoriasis, autoimmune disorders, atherosclerosis, rheumatoid arthritis, osteoarthritis, heart failure, chronic pain, and neuropathic pain.

### BACKGROUND OF THE INVENTION

Mitogen-activated protein kinase (MAPK)/Extracellular signal-regulated protein kinase (Erk) Kinase (“MEK”, or “Mek”) is a dual specificity kinase of the intracellular signaling system in which cell membrane activated Ras proteins transduce signals in a coupled cascade to control cytosolic and nuclear processes (Sebolt-Leopold J. S. and Herrera, R., Nat Rev Cancer, 4:937-947, 2004; Kolch, W., Nat Rev Mol Cell Biol, 6:827-837, 2005). The two Mek isoforms, Mek1 and Mek2, are involved in proliferative diseases such as cancer and vascular restenosis, immunomodulation, and inflammation. Defects in intracellular signal transduction in which Mek participates underlie many of the mechanistic aspects of tumor growth, immune dysfunction, and hyper-inflammatory conditions.

These defects include a change either in the intrinsic activity or in the cellular concentration of one or more proteins in a signaling cascade. For example, a cell may produce a growth factor that binds to its own receptors, resulting in an autocrine signaling loop (Mosesson, Y. and Yarden, Y., Semin Cancer Biol, 14:262-270, 2004). Mutations or over-expression of intracellular signaling proteins can also lead to spurious activating signals. In cancer, some of the most common mutations occur in genes encoding proteins of the Ras family, small monomeric G-proteins that are activated when bound to GTP, and inactivated when bound to GDP (Friday, B. B. and Adjei, A. A., Biochim Biophys Acta, 25:127-144, 2005). Ligand-bound receptor tyrosine kinases, and other mitogenic receptors, stimulate membrane proximal events that convert Ras proteins from the GDP-bound state to the GTP-bound state. This signal is a critical prerequisite for proliferation in most cell types (Stacey D. W., et al., Oncogene, 6:2297-2304, 1991; Takuwa N. and Takuwa, Y., Mol Cell Endocrinol, 177:25-33, 2001; Tuveson, D. A., et al., Cancer Cell, 5:375-87, 2004). Ras mutations that inhibit the deactivation of the Ras-GTP complex lead to chronic stimulation of downstream components. These mutations are found in about 30% of all human cancers, and with much higher prevalence in particular diseases such as colorectal carcinoma, non small-cell lung carcinoma, and colorectal carcinoma (Bos, J. L., Cancer Res, 49:4682-4689, 1989; Hoshino, R. et. al., Oncogene, 18:813-822, 1999).

Activated Ras induces phosphorylation events that activate Raf kinases, which in turn phosphorylate and activate Mek (i.e., Mek1 and Mek2). Activated Mek then phosphorylates and activates the MAP kinases Erk1 and Erk2 (Sebolt-Leopold, J. S. and Herrera, R., Nat Rev Cancer, 4:937-947, 2004). In fact, Mek is the only known kinase that activates Erk1 and Erk2. Constitutively active Mek mutants are sufficient to induce cellular transformation and to force tumor formation in vivo, demonstrating that Mek is, experimentally, an oncogene (Cowley, S., et al., Cell, 77:841-52, 1994; Mansour, S. J., et al., Science, 265:966-970, 1994; Greulich, H. and Erikson, R. L., J Biol Chem, 273:13280-13288, 1998). Blockade of Ras signaling, for example, by use of a trans-dominant mutant Mek1 protein, can block mitogenic signaling, whether induced from cell surface receptors or from oncogenic Ras mutants (Cowley, S., et al., Cell, 77:841-52, 1994).

Raf kinases phosphorylate Mek on two closely adjacent serine residues, S²¹⁸ and S²²² in the case of Mek1. Mek specific activity is very low in the absence of at least one of these phosphorylations, and increases more than 1000 fold in their presence (Huang, W., et al., Mol Biol Cell, 6:237-245, 1995). Mek in turn phosphorylates and activates Erk (on both a tyrosine, Y¹⁸⁵, and a threonine, T¹⁸³, in Erk1). Activated Erk then phosphorylates a large number of cytosolic and nuclear proteins, as active dimeric Erk translocates to the cell nucleus (Lenormand, P., et al., J Cell Biol, 142:625-633, 1998). These phosphorylations substantially modulate, generally stimulating, the activity of the target proteins. Mek is exquisitely selective; no substrate for Mek other than Erk1 and Erk2 has been demonstrated to date. Mek does not even phosphorylate peptides based on its Erk phosphorylation motif or phosphorylate denatured Erk. Mek also associates strongly with Erk, suggesting that phosphorylation requires the formation of a Mek-Erk complex (Bardwell, A. J., et al., J Biol Chem, 276:10374-10386, 2000).

The MAPK pathway from Ras to Erk has been the focus of drug discovery, particularly in cancer, for many years (Sebolt-Leopold, J. S., Oncogene, 19:6564-6599, 2000). Several classes of small molecules are known to be relatively selective Mek inhibitors. These compounds inhibit mutant Ras and Raf mediated cell transformation, Erk activation and dependent processes, cell proliferation in vitro, and tumor growth in vivo (Mallon, R., et al., Mol Cancer Ther, 3:755-762, 2004; Sebolt-Leopold, J. S., Curr Pharm Des, 10: 1907-1914, 2004; Sebolt-Leopold J. S. and Herrera, R., Nat Rev Cancer, 4:937-947, 2004). Structural biology and biochemical data for some of these compounds are consistent with an allosteric, non-ATP competitive mechanism of action (Duncia, J. V., et al., Bioorg Med Chem Lett, 8:2839-2844, 1998; Ohren, J. F., et al., Nat Struct Mol Biol, 11:1192-1197, 2004; Han S., et al., Bioorg Med Chem Lett, 15:5467-5473, 2005; Wallace, E. M., et al., Curr Top Med Chem, 5:215-29, 2005). These observations support the notion of Mek as an attractive drug target, with the opportunity of a high degree of target selectivity.

It has been found that the compounds of the present invention are inhibitors of Mek and are useful in the treatment of a variety of proliferative disease states, such as various cancers, as well as immunological and inflammatory diseases modulated by the Ras-Raf-Mek-Erk cascade.

### SUMMARY OF THE INVENTION

Novel compounds and pharmaceutical compositions that treat cancer, non-cancer hyperproliferative disorders, vascular restenosis, psoriasis, autoimmune disorders, atherosclerosis, rheumatoid arthritis, osteoarthritis, heart failure, chronic pain, and neuropathic pain by inhibiting MEK have been found together with methods of synthesizing and using the compounds including methods for inhibiting MEK in a patient by administering the compounds.

The present invention discloses a class of compounds, usefuil in treating MEK-mediated disorders and conditions, defined by structural Formula I: 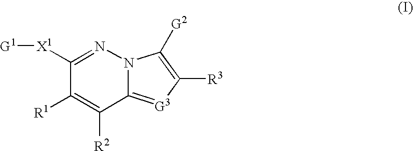
or a salt, ester, or prodrug thereof, wherein:

G¹ is selected from the group consisting of alkyl, aminoalkyl, hydroxyalkyl, haloalkyl, perhaloalkyl, acyl, alkenyl, alkynyl, heteroalkyl, cycloalkyl, cycloalkylalkyl, heterocycloalkyl, aryl, arylalkyl, arylalkenyl, arylalkynyl, heteroaryl, heteroarylalkyl, heteroarylalkenyl, and heteroarylalkynyl, any of which may be optionally substituted;

R¹ and R² are independently selected from the group consisting of hydrogen, acylamino, alkanoyl, alkyl, alkenyl, alkynyl, alkoxy, alkoxyalkyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

R³ is selected from the group consisting of hydrogen, alkanoyl, alkyl, alkenyl, alkynyl, alkoxy, alkoxyalkyl, alkoxycarbonyl, alkoxycarbonylalkyl, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, aminoalkyl, aminocarbonyl, aminocarbonylalkyl, arylalkoxy, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

X¹ is selected from the group consisting of a bond, —N(R⁴)—, —O—, —S—, —SO—, —SO₂—, alkylene, alkenylene, perhaloalkylene, —C(═O)—, —C(═O)C(═O)—, —C(═O)O—, —C(═O)N(R⁵)—, —N(R⁵)C(═O)N(R⁶)—, —N(R⁵)SO₂N(R⁶)—, —N(R⁵)C(═O)O—, —C(═O)[C(R⁷)(R⁸)]ₘ—, —C(═O)O[C(R⁷)(R⁸)]ₘ—, —SO₂N(R⁵)—, —C(═S)—, —C(═O)S—, —S(═O)ₙ[C(R⁷)(R⁸)]ₘ—, —P(═O)(OR⁹)—, —P(═O)(NR⁹)—, —P(═S)(OR⁹)—, —P(═S)(NR⁹)—, —S(═O)(═NR⁹)—, and —S(═NR⁹)(—NR⁹)—, any of which may be optionally substituted;

R⁴, R⁵, R⁶, R⁷, R⁸, and R⁹are independently selected from the group consisting of hydrogen, acyl, alkoxycarbonyl, alkoxycarbonylalkyl, alkyl, alkylsulfonyl, alkenyl, alkynyl, alkoxyalkyl, alkylaminoalkyl, alkylcarbonyl, amido, aminoalkyl, aryl, arylalkyl, arylalkenyl, arylalkynyl, cycloalkyl, cycloalkylalkyl, haloalkyl, perhaloalkyl, heteroaryl, heteroarylalkyl, heteroarylalkenyl, and heterocycloalkyl, any of which may be optionally substituted; or R⁵ and R⁶, together with the atoms to which they are attached, may be joined to form an optionally substituted cycloalkyl or optionally substituted heterocycloalkyl moiety; or R⁷ and R⁸, together with the atoms to which they are attached, may be joined to form an optionally substituted cycloalkyl or optionally substituted heterocycloalkyl moiety;

G² is selected from a structure from the group consisting of:

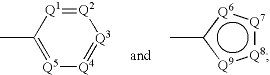

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R³)—, —N—, —N(R¹⁶)—, —O—, and —S—;

Q⁹ is selected from the group consisting of —C(R⁷)—, —N—, —N(R¹⁸)—, —O—, and —S—;

R¹⁰, R¹³, R¹⁴, R¹⁷ and R¹⁸ are independently selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl and cycloalkylalkyl, any of which may be optionally substituted;

R¹¹ is selected from the group consisting of hydrogen, fluoro, bromo, iodo, cyano, nitro, hydroxy, acylamino, alkyl, alkylamino, alkoxy, alkoxyalkoxy, haloalkyl, perhaloalkyl, haloalkoxy, aryl, aralkyl, arylalkenyl, arylalkynyl, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylalkylamino, heteroaryloxy, heteroarylalkoxy, arylthio, arylsulfonyl, heteroarylthio, heteroarylalkylthio, heterocycloalkyl, cycloalkyl, and cycloalkylalkyl, any of which may be optionally substituted;

R¹² is selected from the group consisting of hydrogen, fluoro, bromo, iodo, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl, and cycloalkylalkyl, any of which may be optionally substituted;

R¹⁵ is selected from the group consisting of hydrogen, halogen, cyano, nitro, hydroxy, acyl, acylamino, alkyl, alkylamino, haloalkyl, perhaloalkyl, aryl, aralkyl, arylalkenyl, arylalkynyl, arylamino, arylalkylamino, arylthio, arylsuilfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, arylthio, arylsulfonyl, heteroarylthio, heteroarylalkylthio, heterocycloalkyl, cycloalkyl, and cycloalkylalkyl, any of which may be optionally substituted;

R¹⁶ is selected from the group consisting of hydrogen, acyl, alkyl, perhaloalkyl, haloalkoxy, aralkyl, arylalkenyl, arylalkynyl, arylsulfonyl, alkenyl, alkynyl, alkoxycarbonyl, alkoxycarbonylalkyl, alkylaminocarbonyl, alkylaminocarbonylalkyl, alkanoyl, hydroxyalkyl, alkylsulfonyl, heteroarylalkyl, heteroarylalkenyl, heteroarylalkynyl, arylsulfonyl, heterocycloalkyl, cycloalkyl, and cycloalkylalkyl, any of which may be optionally substituted;

G³ is selected from the group consisting of —N— and C(R¹⁹);

R¹⁹ is selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, and heteroarylalkylthio, any of which may be optionally substituted;

m is an integer from 1 to 5;

n is an integer from 0 to 2;

and provided that when X¹ is —NH—, and G¹ is 2-thienylmethyl, then G² may not be phenyl.

The present invention discloses a class of compounds, useful in treating MEK-mediated disorders and conditions,,defined by structural Formula II: 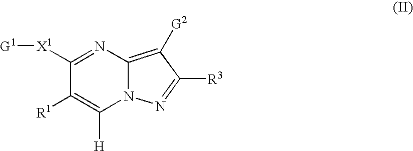
or a salt, ester, or prodrug thereof, wherein:

G¹ is selected from the group consisting of alkyl, aminoalkyl, hydroxyalkyl, haloalkyl, perhaloalkyl, acyl, alkenyl, alkynyl, heteroalkyl, cycloalkyl, cycloalkylalkyl, heterocycloalkyl, aryl, arylalkyl, arylalkenyl, arylalkynyl, heteroaryl, heteroarylalkyl having five ring atoms, heteroarylalkenyl, and heteroarylalkynyl, any of which may be optionally substituted;

R¹ is selected from the group consisting of hydrogen, acylamino, alkanoyl, alkyl, alkenyl, alkynyl, alkoxy, alkoxyalkyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

R³ is selected from the group consisting of from the group consisting of hydrogen, alkanoyl, alkyl, alkenyl, alkynyl, alkoxy, alkoxyalkyl, alkoxycarbonyl, alkoxycarbonylalkyl, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amidb, aminoalkyl, aminocarbonyl, aminocarbonylalkyl, arylalkoxy, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxyalkyl, nitro, and thiol, any of which may be optionally substituted;

X¹ is selected from the group consisting of a bond, —N(R⁴)—, —O—, —S—, —SO—, —SO₂—, alkylene, alkenylene, perhaloalkylene, —C(═O)—, —C(═O)C(═O)—, —C(═O)O—, —C(═O)N(R⁵)—, —N(R⁵)C(═O)N(R⁶)—, —N(R⁵)SO₂N(R⁶)—, —N(R⁵)C(═O)O—, —C(═O)[C(R⁷)(R⁸)]ₘ, —C(═O)O[C(R⁷)(R⁸)]ₘ, —SO₂N(R⁵)—, —C(═S)—, —C(═O)S—, —S(═O)ₙ[C(R⁷)(R⁸)]ₘ—, —P(═O)(OR⁹)—, —P(═O)(NR⁹)—, —P(═S)(OR⁹)—, —P(═S)(R⁹)—, —S(═O)(═NR⁹)—, and —S(═NR⁹)(═NR⁹)—, any of which may be optionally substituted;

R⁴, R⁵, R⁶, R⁷, R⁸, and R⁹ are independently selected from the group consisting of hydrogen, acyl, alkoxycarbonyl, alkoxycarbonylalkyl, alkyl, alkylsulfonyl, alkenyl, alkynyl, alkoxyalkyl, alkylaminoalkyl, alkylcarbonyl, amido, aminioalkyl, aryl, arylalkyl, arylalkenyl, arylalkynyl, cycloalkyl, cycloalkylalkyl, haloalkyl, perhaloalkyl, heteroaryl, heteroarylalkyl, heteroarylalkenyl, and heterocycloalkyl, any of which may be optionally substituted; or R⁵ and R⁶, together with the atoms to which they are attached, may be joined to form an optionally substituted cycloalkyl or optionally substituted heterocycloalkyl moiety; or R⁷ and R⁸, together with the atoms to which they are attached, may be joined to form an optionally substituted cycloalkyl or optionally substituted heterocycloalkyl moiety;

G² is selected from heteroaryl and a structure consisting of:

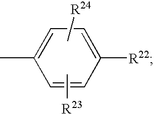

wherein:

R²² is selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, heterocycloalkyl, cycloalkyl and cycloalkylalkyl, any of which may be optionally substituted;

R²³ and R²⁴are independently selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl and cycloalkylalkyl, any of which may be optionally substituted;

m is an integer from 1 to 5; and

n is an integer from 0 to 2.

The present invention discloses a class of compounds, useful in treating MEK-mediated disorders and conditions, defined by structural Formula III: 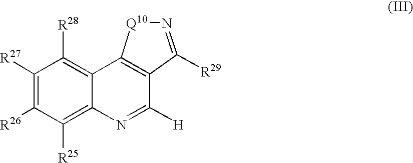
or a salt, ester, or prodrug thereof, wherein:

R²⁵ is selected from the group consisting of hydrogen, acylamino, alkanoyl, alkyl, alkenyl, alkynyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

R²⁶ and R²⁸ are independently selected from the group consisting of hydrogen, acylamino, alkanoyl, alkyl, alkenyl, alkynyl, alkoxy, alkoxyalkyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

R²⁷ is selected from the group consisting of hydrogen, acylamino, alkanoyl, alkyl, alkenyl, alkynyl, alkoxy, alkoxyalkyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

R²⁹ is selected from the group consisting of acylamino, alkyl, alkenyl, alkynyl, alkoxy, alkoxyalkyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylthio, alkylsulfonyl, amino, aminoalkyl, arylalkoxy, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;

Q¹⁰ is selected from the group consisting of —N(R³⁰)—, —O—, and —S—; and

R³⁰ is selected from the group consisting of aryl, heteroaryl, cycloalkyl and heterocycloalkyl, any of which may be optionally substituted.

The present invention discloses a class of compounds, useful in treating MEK-mediated disorders and conditions, defined by structural Formula IV: 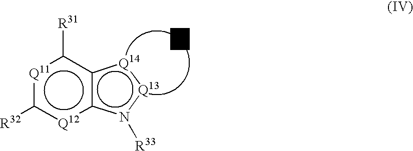
or a salt, ester, or prodrug thereof, wherein:

A is a carbocyclic, heterocyclic, aromatic, or heteroaromatic group, any of which may be optionally substituted and each of which have five to eight ring atoms including Q¹³ and Q¹⁴; or, alternatively, A may be absent;

R³¹ is selected from the group consisting of hydrogen and —N(R³⁴)(R³⁵);

R³² is selected from the group consisting of hydrogen and hydroxy;

R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;

R³⁴ and R³⁵ are independently selected from the group consisting of hydrogen, acyl, alkyl, alkenyl, alkynyl, alkoxyalkyl, alkylaminoalkyl, alkylcarbonyl, amido, aminoalkyl, aryl, arylalkyl, cycloalkyl, cycloalkylalkyl, perhaloalkyl, heteroaryl, heteroarylalkyl, and heterocycloalkyl, any of which may be optionally substituted; or R³⁴and R³⁵, together with the atoms to which they are attached, may be joined to form an optionally substituted heterocycloalkyl moiety;

Q¹¹ is selected from the group consisting of —N— and —C(R³⁶)—;

Q¹² is selected from the group consisting of —N— and —C(R³⁷)—;

Q¹³ is selected from the group consisting of —N—, and —C(R³⁸)—;

Q¹⁴ is selected from the group consisting of —N— and —(R³⁹)—;

R³⁶ and R³⁷ are independently selected from the group consisting of hydrogen, alkyl, alkenyl, and alkynyl, any of which may be optionally substituted; and

R³⁸ and R³⁹ are independently selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted.

Compounds according to the present invention possess useful MEK inhibiting activity, and may be used in the treatment or prophylaxis of a disease or condition in which MEK plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for inhibiting or modulating MEK. In other embodiments, the present invention provides methods for treating a MEK-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the inhibition of MEK activity.

In other aspects, the present invention provides methods for treating MEK-related disorders in a human or animal subject in need of such treatment comprising administering to said subject an amount of a compound of formula (1) effective to reduce or prevent tumor growth in the subject.

In yet other aspects, the present invention provides methods for treating MEK-related disorders in a human or animal subject in need of such treatment comprising administering to said subject an amount of a compound of Formulas (I) through (IV) effective to reduce or prevent tumor growth in the subject in combination with at least one additional agent for the treatment of cancer as known by those skilled in the art.

In yet other aspects, the present invention provides therapeutic compositions comprising at least one compound of Formulas (I) through (IV), in combination with one or more additional agents for the treatment of cancer, as are commonly employed in cancer therapy. Both the compound or compounds of any one or more of Formulas (I) through (IV) and the additional agent may be put into the same dosage form, or administered separately.

The compounds of the invention are useful in the treatment of cancer, non-cancer hyperproliferative disorders, vascular restenosis, psoriasis, autoimmune disorders, atherosclerosis, rheumatoid arthritis, osteoarthritis, heart failure, chronic pain, and neuropathic pain.

The invention further provides compositions, methods of use, and methods of manufacture as described in the detailed description of the invention.

### DETAILED DESCRIPTION OF THE INVENTION

In certain embodiments, the compounds of the present invention have structural Formula I wherein:

G¹ is selected from the group consisting of alkyl, aminoalkyl, hydroxyalkyl, perhaloalkyl, acyl, alkenyl, alkynyl, heteroalkyl, cycloalkyl, cycloalkylalkyl, heterocycloalkyl, aryl, arylalkyl, heteroaryl, and heteroarylalkyl, any of which may be optionally substituted;

R¹ and R² are independently selected from the group consisting of hydrogen, acylamino, alkyl, alkoxy, alkoxycarbonyl, alkoxycarbonylalkyl, alkylaminocarbonyl, alkylaminocarbonylalkyl, alkoxyaminocarbonyl, alkoxyaminocarbonylalkyl, alkylcarbonyl, alkylthio, alkylsulfonyl, amido, aminocarbonyl, aminocarbonylalkyl, carboxy, cyano, cycloalkyl, cycloalkylalkyl, halogen, perhaloalkoxy, perhaloalkyl, heterocycloalkyl, and nitro, any of which may be optionally substituted;

R³ is selected from the group consisting of hydrogen, alkyl, alkoxy, alkoxyalkyl, alkoxycarbonyl, alkoxycarbonylalkyl, alkylaminocarbonyl, alkylaminocarbonylalkyl, alkylcarbonyl, alkylthio, alkylsulfonyl, aminocarbonyl, aminocarbonylalkyl, carboxy, cyano, cycloalkyl, cycloalkylalkyl, halogen, perhaloalkoxy, perhaloalkyl, hydroxy, hydroxyalkyl, nitro, and thiol, any of which may be optionally substituted;

X¹ is selected from the group consisting of —N(R⁴)—, —O—, —S—, —SO₂—, alkylene, alkenylene, —C(═O)O—, —C(═O)N(R⁵)—, —N(R⁵)C(═O)N(⁶)—, —N(R⁵)SO₂N(R⁶)—, —N(R⁵)C(═O)O—, —C(═O)O[C(R⁷)(R⁸)]ₘ—, —SO₂N(R⁵)—, and —S(═O)ₙ[C(R⁷)(R⁸)]ₘ, any of which may be optionally substituted;

G2 is selected from a structure from the group consisting of:

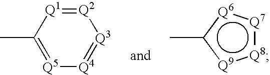

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁸)—, —O—, and —S—;

R⁴, R⁵, R⁶, R⁷, and R⁸are independently selected from the group consisting of hydrogen, acyl, alkoxycarbonyl, alkoxycarbonylalkyl, alkyl, alkylsulfonyl, alkenyl, alkynyl, alkoxyalkyl, alkylaminoalkyl, alkylcarbonyl, arylalkyl, amido, aminoalkyl, cycloalkyl, cycloalkylalkyl, perhaloalkyl, heteroarylalkyl, and heterocycloalkyl, any of which may be optionally substituted; or R⁵ and R⁶, together with the atoms to which they are attached, may be joined to form an optionally substituted cycloalkyl or optionally substituted heterocycloalkyl moiety; or R⁷ and R⁸, together with the atoms to which they are attached, may be joined to form an optionally substituted cycloalkyl or optionally substituted heterocycloalkyl moiety;

R¹⁰, R¹³, R¹⁴, R¹⁷, and R¹⁸ are independently selected from the group consisting of hydrogen, acyl, alkoxy, alkoxycarbonyl, alkyl, halogen, cyano, carboxylate, acylamino, alkylamino, perhaloalkyl, aralkyl, alkenyl, alkynyl, alkoxycarbonylalkyl, alkylaminocarbonyl, alkylaminocarbonylalkyl, alkylthio, alkylsulfonyl, alkylsulfonylamino, heteroarylalkyl, heterocycloalkyl, cycloalkyl, and cycloalkylalkyl, any of which may be optionally substituted;

R¹¹ is selected from the group consisting of hydrogen, fluoro, bromo, iodo, cyano, acylamino, alkyl, alkoxy, perhaloalkyl, haloalkoxy, aralkyl, aryloxy, arylalkoxy, arylthio, arylsulfonyl, alkoxycarbonyl, alkoxycarbonylalkyl, alkylaminocarbonyl, alkylaminocarbonylalkyl, alkylthio, alkylsulfonyl, alkylsulfonylamino, carboxylate, heteroarylalkyl, heterocycloalkyl, cycloalkyl, and cycloalkylalkyl, any of which may be optionally substituted;

R¹² is selected from the group consisting of hydrogen, fluoro, bromo, iodo, cyano, acylamino, alkyl, alkylamino, alkoxy, perhaloalkyl, aralkyl, arylamino, arylalkylamino, aryloxy, arylalkoxy, arylthio, arylsulfonyl, alkoxycarbonyl, alkoxycarbonylalkyl, alkylaminocarbonyl, alkylaminocarbonylalkyl, alkylthio, alkylsulfonyl, alkylsulfonylamino, heteroarylalkyl, heteroarylamino, heteroarylalkylamino, heteroaryloxy, heteroarylalkoxy, arylthio, arylsulfonyl, heteroarylthio, heteroarylalkylthio, heterocycloalkyl, carboxy, cycloalkyl, and cycloalkylalkyl, any of which may be optionally substituted;

R¹⁵ is selected from the group consisting of acyl, alkoxycarbonyl, alkyl, halogen, cyano, carboxylate, acylamino, alkylamino, perhaloalkyl, aralkyl, arylamino, arylalkylamino, arylthio, arylsulfonyl, alkoxycarbonylalkyl, alkylaminocarbonyl, alkylaminocarbonylalkyl, alkylthio, alkylsulfonyl, alkylsulfonylamino, heteroarylalkyl, heteroarylamino, heteroarylalkylamino, arylthio, arylsulfonyl, heteroarylthio, heteroarylalkylthio, heterocycloalkyl, cycloalkyl, and cycloalkylalkyl, any of which may be optionally substituted;

R¹⁶ is selected from the group consisting of hydrogen, acyl, alkoxycarbonyl, alkyl, perhaloalkyl, aralkyl, arylsulfonyl, alkoxycarbonylalkyl, alkylaminocarbonyl, alkylaminocarbonylalkyl, alkylsulfonyl, heteroarylalkyl, arylsulfonyl, heterocycloalkyl, cycloalkyl, and cycloalkylalkyl, any of which may be optionally substituted;

G³ is —N—;

m is an integer from 1 to 2; and

n is the integer 2.

In further embodiments, the compounds of the present invention have structural Formula I wherein:

G¹ is selected from the group consisting of alkyl, aminoalkyl, hydroxyalkyl, cycloalkyl, cycloalkylalkyl, heterocycloalkyl, aryl, arylalkyl, heteroaryl, and heteroarylalkyl, any of which may be optionally substituted;

R¹ and R² are independently selected from the group consisting of hydrogen, alkyl, alkoxy, alkylaminocarbonyl, alkylaminocarbonylalkyl, alkoxyaminocarbonyl, alkoxyaminocarbonylalkyl, alkylcarbonyl, alkylthio, amido, aminocarbonyl, aminocarbonylalkyl, cyano, cycloalkyl, cycloalkylalkyl, halogen, and perhaloalkyl, any of which may be optionally substituted;

R³ is selected from the group consisting of from the group consisting of hydrogen, alkyl, alkoxy, alkylthio, alkylsulfonyl, cyano, cycloalkyl, cycloalkylalkyl, halogen, and perhaloalkyl, any of which may be optionally substituted;

X¹ is selected from the group consisting of —N(R⁴)—, —O—, —S—, —SO₂—, —C(═O)N(R⁵)—, —N(R⁵)C(═O)O—, and —SO₂N(R⁵)—, any of which may be optionally substituted;

R⁴ and R⁵ are independently selected from the group consisting of hydrogen, acyl, alkoxycarbonyl, alkoxycarbonylalkyl, alkyl, alkylsulfonyl, alkylcarbonyl, arylalkyl, heteroarylalkyl, cycloalkyl, and cycloalkylalkyl, any of which may be optionally substituted;

G² is selected from a structure from the group consisting of:

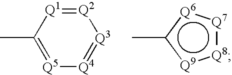

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁸)—, —O—, and —S—;

R¹⁰, R¹³, R¹⁴, R¹⁷, and R¹⁸ are independently selected from the group consisting of hydrogen, acyl, alkoxy, alkoxycarbonyl, alkyl, halogen, cyano, carboxylate, acylamino, alkyl, alkylamino, perhaloalkyl, alkylthio, alkylsulfonyl, alkylsulfonylamino, heteroarylalkyl, cycloalkyl, and cycloalkylalkyl, any of which may be optionally substituted;

R¹¹ is selected from the group consisting of hydrogen, fluoro, bromo, cyano, acylamino, alkyl, alkoxy, perhaloalkyl, alkoxycarbonyl, alkoxycarbonylalkyl, alkylaminocarbonyl, alkylaminocarbonylalkyl, alkylthio, alkylsulfonyl, alkylsulfonylamino, carboxylate, cycloalkyl, and cycloalkylalkyl, any of which may be optionally substituted;

R¹² is selected from the group consisting of hydrogen, fluoro, bromo, cyano, acylamino, alkyl, alkylamino, alkoxy, perhaloalkyl, arylamino, arylalkylamino, aryloxy, arylalkoxy, arylthio, arylsulfonyl, alkoxycarbonyl, alkylaminocarbonyl, alkylthio, alkylsulfonyl, alkylsulfonylamino, heteroarylamino, heteroarylalkylamino, heteroaryloxy, heteroarylalkoxy, arylthio, arylsulfonyl, heteroarylthio, heteroarylalkylthib, carboxy, cycloalkyl, and cycloalkylalkyl, any of which may be optionally substituted;

R¹⁵ is selected from the group consisting of hydrogen, acyl, alkoxycarbonyl, alkyl, halogen, cyano, carboxylate, perhaloalkyl, alkoxycarbonylalkyl, alkylaminocarbonyl, alkylaminocarbonylalkyl, alkylthio, alkylsulfonyl, alkylsuilfonylamino, cycloalkyl, and cycloalkylalkyl, any of which may be optionally substituted; and

R¹⁶ is selected from the group consisting of hydrogen, acyl, alkoxycarbonyl, alkyl, alkoxycarbonylalkyl, alkylaminocarbonyl, alkylaminocarbonylalkyl, alkylsulfonyl, arylsulfonyl, cycloalkyl, and cycloalkylalkyl, any of which may be optionally substituted.

In further embodiments, the compounds of the present invention have structural Formula I wherein:

G¹ is selected from the group consisting of alkyl, aminoalkyl, hydroxyalkyl, perhaloalkyl, acyl, alkenyl, alkynyl, heteroalkyl, cycloalkyl, cycloalkylalkyl, heterocycloalkyl, aryl, arylalkyl, heteroaryl, and heteroarylalkyl, any of which may be optionally substituted;

R¹ and R² are independently selected from the group consisting of hydrogen, alkyl, alkylaminocarbonyl, alkylaminocarbonylalkyl, alkoxyaminocarbonyl, alkoxyaminocarbonylalkyl, aminocarbonyl, aminocarbonylalkyl, cyano, cycloalkyl, cycloalkylalkyl, and halogen, any of which may be optionally substituted;

R³ is selected from the group consisting of hydrogen, alkyl, cycloalkyl, cycloalkylalkyl, and perhaloalkyl, any of which may be optionally substituted;

X¹ is selected from the group consisting of —N(R⁴)—, —O—, and —S—, any of which may be optionally substituted;

R⁴ is selected from the group consisting of hydrogen, alkyl, cycloalkyl, and cycloalkylalkyl, any of which may be optionally substituted;

G² is selected from a structure from the-group consisting of:

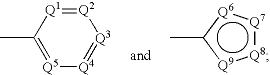

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁸)—, —O—, and —S—;

R¹⁰, R¹³, R¹⁴, R¹⁷, and R¹⁸ are independently selected from the group consisting of hydrogen, acyl, alkoxy, alkoxycarbonyl, alkyl, halogen, cyano, carboxylate, alkylthio, cycloalkyl, and cycloalkylalkyl, any of which may be optionally substituted;

R¹¹ is selected from the group consisting of hydrogen, fluoro, bromo, cyano, alkyl, alkoxy, alkoxycarbonyl, carboxylate, alkylthio, cycloalkyl, and cycloalkylalkyl, any of which may be optionally substituted; and

R¹² is selected from the group consisting of hydrogen, fluoro, bromo, cyano, alkyl, alkoxy, alkoxycarbonyl, alkylthio, carboxy, cycloalkyl, and cycloalkylalkyl, any of which may be optionally substituted.

In further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro-4-iodophenyl;

R¹ and R²are independently selected from the group consisting of hydrogen, alkyl, alkoxyaminocarbonyl, and alkoxyaminocarbonylalkyl, any of which may be optionally substituted;.

R³ is selected from the group consisting of the group consisting of hydrogen and alkyl, any of which may be optionally substituted;

X¹ is —N(R⁴)—;

R⁴ is selected from the group consisting of hydrogen, alkyl, cycloalkyl, and cycloalkylalkyl, any of which may be optionally substituted;

G² is selected from a structure from-the group consisting of:

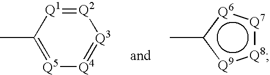

wherein:

Q¹ and Q⁵ are independently selected from the group consisting of—C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁸)—, —O—, and —S—;

R¹⁰, R¹³, R¹⁴, R¹⁷ , and R¹⁸ are independently selected from the group consisting of hydrogen, acyl, alkoxy, alkoxycarbonyl, alkyl, halogen, cyano, carboxylate, alkylthio, cycloalkyl, and cycloalkylalkyl, any of which may be optionally substituted;

R¹¹ is selected from the group consisting of hydrogen, fluoro, bromo, cyano, alkyl, alkoxy, alkoxycarbonyl, carboxylate, alkylthio, cycloalkyl, and cycloalkylalkyl, any of which may be optionally substituted; and

R¹² is selected from the group consisting of hydrogen, fluoro, bromo, cyano, alkyl, alkoxy, alkoxycarbonyl, alkylthio, carboxy, cycloalkyl, and cycloalkylalkyl, any of which may be optionally substituted.

In further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro4-iodophenyl;

R¹ is selected from the group consisting of hydrogen, methyl, ethyl, N-(2-hydroxyethoxy)-carboxamido, and N-(2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² is selected from a structure from the group consisting of:

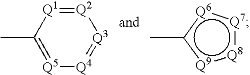

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of—C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁸)—, —O—, and —S—;

R¹⁰, R¹³, R¹⁴, R¹⁷ and R¹⁸ are independently selected from the group consisting of hydrogen, acyl, alkoxy, alkoxycarbonyl, alkyl, halogen, cyano, carboxylate, alkylthio, cycloalkyl, and cycloalkylalkyl, any of which may be optionally substituted;

R¹¹ is selected from the group consisting of hydrogen, fluoro, bromo, cyano, alkyl, alkoxy, alkoxycarbonyl, carboxylate, alkylthio, cycloalkyl, and cycloalkylalkyl, any of which may be optionally substituted; and

R¹² is selected from the group consisting of hydrogen, fluoro, bromo, cyano, alkyl, alkoxy, alkoxycarbonyl, alkylthio, carboxy, cycloalkyl, and cycloalkylalkyl, any of which may be optionally substituted.

In further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-(2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² has the structure:

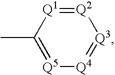

wherein:

Q¹ and Q⁵ are independently selected from the group consisting of —C(R¹⁰)—, and —N—;

Q² and Q⁴ are independently selected from the group consisting of —C(R¹¹)—, and —N—;

Q³ is selected from the group consisting of —C(R¹²)— and —N—;

Q⁶ is selected from the group consisting of —C(R¹³)—, —N—, —N(R¹⁴)—, —O—, and —S—;

Q⁷ is selected from the group consisting of —C(R¹⁵)—, —N—, —N(R¹⁶)—, —O—, and —S—;

Q⁸ is selected from the group consisting of —C(R¹³)—, —N—, —N(R¹⁶)—, —O—, and —S—;

Q⁹ is selected from the group consisting of —C(R¹⁷)—, —N—, —N(R¹⁷)—, —O—, and —S—;

R¹⁰, R¹¹, R¹⁴, R¹⁷, and R¹⁸ are independently selected from the group consisting of hydrogen, acyl, alkoxy, alkoxycarbonyl, alkyl, halogen, cyano, carboxylate, alkylthio, cycloalkyl, and cycloalkylalkyl, any of which may be optionally substituted;

R¹¹ is selected from the group consisting of hydrogen, fluoro, bromo, cyano, alkyl, alkoxy, alkoxycarbonyl, carboxylate, alkylthio, cycloalkyl, and cycloalkylalkyl, any of which may be optionally substituted; and

R¹² is selected from the group consisting of hydrogen, fluoro, bromo, cyano, alkyl, alkoxy, alkoxycarbonyl, alkylthio, carboxy, cycloalkyl, and cycloalkylalkyl, any of which may be optionally substituted.

In further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2—N,N)-dimethylaminoethyl, propyl, 2-chloro-4-bromophenyl, 2-fluoro4-bromophenyl, and 2-fluoro-4-idophenyl;

R¹ is selected from the group consisting of hydrogen, methyl, ethyl, N-2-hydroxyethoxy)-carboxamido, and N-2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² has the structure:

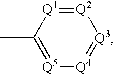

wherein:

Q¹ and Q⁵ are independently selected from the Q¹ and Q⁵ are —C(R¹⁰)—;

Q² and Q⁴ are —C(^{R}¹¹)—;

Q³ is —C(R¹²)—;

R¹⁰ and R¹¹ are hydrogen; and

R¹² is selected from the group consisting of hydrogen, carboxy and cyano.

In yet further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-(2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² has the structure:

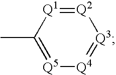

wherein:

Q¹ and Q⁵ are —C(R¹⁰)—;

Q² is —C(R¹¹)—;

Q³ is —C(R¹²)—;

Q⁴ is —N—;

R¹⁰ and R¹² are hydrogen; and

R¹¹ is methoxy.

In yet further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2—N,N)-dimethylaminoethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² has the structure:

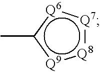

Q⁶ is —S—;

Q⁷ is —C(R¹⁵)—;

Q⁸ is —C(R¹³)—;

Q⁹ is —C(R¹⁷)—;

R¹⁵is acetyl; and

R¹³ and R¹⁷ are hydrogen.

In yet further embodiments, the compounds of the present invention have structural Formula I wherein:

G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-(2,3-dihydroxypropoxy)-carboxamido;

R², R³, and R⁴ are hydrogen;

X¹ is —N(R⁴)—;

R⁴ is selected from the group consisting of hydrogen, alkyl, cycloalkyl, and cycloalkylalkyl, any of which may be optionally substituted;

G² has the structure:

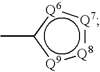

Q⁶ is —C(R¹³)—;

Q⁷ is —N—;

Q⁸ is —N(R¹⁶)—;

Q⁹ is —C(R¹⁷)—;

R¹³ and R¹⁷ are hydrogen; and

R¹⁶ is methyl.

The present invention further discloses a class of compounds, useful in treating MEK-mediated disorders and conditions, defined by structural Formula II wherein:

G¹ is selected from the group consisting of alkyl, aminoalkyl, hydroxyalkyl, perhaloalkyl, acyl, alkenyl, alkynyl, heteroalkyl, cycloalkyl, cycloalkylalkyl, heterocycloalkyl, aryl, arylalkyl, heteroaryl, and heteroarylalkyl, any of which may be optionally substituted;

R¹ is selected from the group consisting of hydrogen, acylamino, alkyl, alkoxy, alkoxycarbonyl, alkoxycarbonylalkyl, alkylaminocarbonyl, alkylaminocarbonylalkyl, alkoxyaminocarbonyl, alkoxyaminocarbonylalkyl, alkylcarbonyl, alkylthio, alkylsulfonyl, amido, aminocarbonyl, aminocarbonylalkyl, carboxy, cyano, cycloalkyl, cycloalkylalkyl, halogen, perhaloalkoxy, perhaloalkyl, heterocycloalkyl, and nitro, any of which may be optionally substituted;

R³ is selected from the group consisting of hydrogen, alkyl, alkoxy, alkoxyalkyl, alkoxycarbonyl, alkoxycarbonylalkyl, alkylaminocarbonyl, alkylaminocarbonylalkyl, alkylcarbonyl, alkylthio, alkylsulfonyl, aminocarbonyl, aminocarbonylalkyl, carboxy, cyano, cycloalkyl, cycloalkylalkyl, halogen, perhaloalkoxy, perhaloalkyl, hydroxyalkyl, nitro, and thiol, any of which may be optionally substituted;

X¹ is selected from the group consisting of —N(R⁴)—, —O—, —S—, —SO₂—, alkylene, alkenylene, —C(═O)O—, —C(═O)N(R⁵)—, —N(R⁵)C(═O)N(R⁶)—, —N(R⁵)SO₂N(R⁶)—, —N(R⁵)C(═O)O—, —C(═O)O[C(R⁷)(R⁸)]ₘ—, —SO₂N(R⁵)—, and —S(═O)ₙ[C(R⁷)(R⁸)]ₘ, any of which may be optionally substituted;

R⁴, R⁵, R⁶, R⁷, and R⁸are independently selected from the group consisting of hydrogen, acyl, alkoxycarbonyl, alkoxycarbonylalkyl, alkyl, alkylsulfonyl, alkenyl, alkynyl, alkoxyalkyl, alkylaminoalkyl, alkylcarbonyl, arylalkyl, amido, aminoalkyl, cycloalkyl, cycloalkylalkyl, perhaloalkyl, heteroarylalkyl, and heterocycloalkyl, any of which may be optionally substituted; or R⁵ and R⁶, together with the atoms to which they are attached, may be joined to form an optionally substituted cycloalkyl or optionally substituted heterocycloalkyl moiety; or R⁷ and R⁸, together with the atoms to which they are attached, may be joined to form an optionally substituted cycloalkyl or optionally substituted heterocycloalkyl moiety;

R²² is selected from the group consisting of hydrogen, acyl, alkoxy, alkoxycarbonyl, alkyl, halogen, cyano, carboxylate, acylamino, alkylamino, perhaloalkyl, aryl, aralkyl, arylamino, arylalkylamino, aryloxy, arylalkoxy, arylsulfonyl, alkoxycarbonylalkyl, alkylaminocarbonyl, alkylaminocarbonylalkyl, alkanoyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, heterocycloalkyl, cycloalkyl and cycloalkylalkyl, any of which may be optionally substituted;

R²³ and R²⁴ are independently selected from the group consisting of hydrogen, acyl, alkoxy, alkoxycarbonyl, alkyl, halogen, cyano, carboxylate, acylamino, alkylamino, alkoxyalkoxy, perhaloalkyl, haloalkoxy, aryl, aralkyl, arylamino, arylalkylamino, aryloxy, arylalkoxy, arylthio, arylsulfonyl, alkoxycarbonylalkyl, alkylaminocarbonyl, alkylaminocarbonylalkyl, alkanoyl, alkylthio, alkylsulfonyl, alkylsulfonylamino, heteroaryl, heteroarylalkyl, heteroarylalkenyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl and cycloalkylalkyl, any of which may be optionally substituted;

m is an integer from 1 to 2; and

n is the integer 2.

In other embodiments, the compounds of the present invention have structural Formula II wherein:

G¹ is selected from the group consisting of alkyl, aminoalkyl, hydroxyalkyl, cycloalkyl, cycloalkylalkyl, heterocycloalkyl, aryl, arylalkyl, heteroaryl, and heteroarylalkyl, any of which may be optionally substituted;

R¹ is selected from the group consisting of hydrogen, alkyl, alkoxy, alkylaminocarbonyl, alkylaminocarbonylalkyl, alkoxyaminocarbonyl, alkoxyaminocarbonylalkyl, alkylcarbonyl, alkylthio, amido, aminocarbonyl, aminocarbonylalkyl, cyano, cycloalkyl, cycloalkylalkyl, halogen, and perhaloalkyl, any of which may be optionally substituted;

R³ is selected from the group consisting of hydrogen, alkyl, alkoxy, alkylthio, alkylsulfonyl, cyano, cycloalkyl, cycloalkylalkyl, halogen, and perhaloalkyl, any of which may be optionally substituted;

X¹ is selected from the group consisting of —N(R⁴)—, —O—, —S—, —SO₂—, —C(═O)N(R⁵)—, —N(R⁵)C(═O)O—, and —SO₂N(R⁵)—, any of which may be optionally substituted;

R⁴ and R⁵ are independently selected from the group consisting of hydrogen, acyl, alkoxycarbonyl, alkoxycarbonylalkyl, alkyl, alkylsulfonyl, alkylcarbonyl, arylalkyl, heteroarylalkyl, cycloalkyl, and cycloalkylalkyl, any of which may be optionally substituted;

G² is selected from heteroaryl and a structure consisting of:

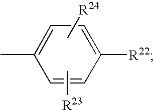

wherein:

R²² is selected from the group consisting of hydrogen, acyl, alkoxy, alkoxycarbonyl, alkyl, halogen, cyano, carboxylate, acylamino, alkylamino, perhaloalkyl, alkoxycarbonylalkyl, alkylaminocarbonyl, alkylaminocarbonylalkyl, alkanoyl, alkylthio, alkylsulfonyl, alkylsulfonylamino, heterocycloalkyl, cycloalkyl and cycloalkylalkyl, any of which may be optionally substituted; and

R²³ and R²⁴are independently selected from the group consisting of hydrogen, acyl, alkoxy, alkoxycarbonyl, alkyl, halogen, cyano, carboxylate, acylamino, alkylamino, alkoxyalkoxy, perhaloalkyl, arylamino, arylalkylamino, aryloxy, arylalkoxy, arylthio, arylsulfonyl, alkoxycarbonylalkyl, alkylaminocarbonyl, alkylaminocarbonylalkyl, alkanoyl, alkylthio, alkylsulfonyl, alkylsulfonylamino, heteroarylamino, heteroarylalkylamino, heteroaryloxy, heteroarylalkoxy, heterocycloalkyl, cycloalkyl and cycloalkylalkyl, any of which may be optionally substituted.

In further embodiments, the compounds of the present invention have structural Formula II wherein:

G¹ is selected from the group consisting of alkyl, aminoalkyl, hydroxyalkyl, cycloalkyl, cycloalkylalkyl, heterocycloalkyl, aryl, arylalkyl, heteroaryl, and heteroarylalkyl, any of which may be optionally substituted;

R¹ is selected from the group consisting of hydrogen, alkyl, alkylaminocarbonyl, alkylaminocarbonylalkyl, alkoxyaminocarbonyl, alkoxyaminocarbonylalkyl, aminocarbonyl, aminocarbonylalkyl, cyano, cycloalkyl, cycloalkylalkyl, and halogen, any of which may be optionally substituted;

R³ is selected from the group consisting of hydrogen, alkyl, cycloalkyl, cycloalkylalkyl, and perhaloalkyl, any of which may be optionally substituted;

X¹ is selected from the group consisting of —N(R⁴)—, —O—, and —S—, any of which may be optionally substituted;

R⁴ is selected from the group consisting of hydrogen, alkyl, cycloalkyl, and cycloalkylalkyl, any of which may be optionally substituted;

G² is selected from heteroaryl and a structure consisting of:

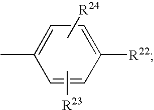

wherein:

R²² is selected from the group consisting of hydrogen, acyl, alkoxy, alkoxycarbonyl, alkyl, halogen, cyano, carboxylate, alkoxycarbonylalkyl, alkanoyl, alkylthio, cycloalkyl and cycloalkylalkyl, any of which may be optionally substituted; and

R²³ and R²⁴ are independently selected from the group consisting of hydrogen, acyl, alkoxy, alkoxycarbonyl, alkyl, halogen, cyano, carboxylate, perhaloalkyl, alkoxycarbonylalkyl, alkylaminocarbonyl, alkylaminocarbonylalkyl, alkanoyl, alkylthio, alkylsulfonyl, alkylsulfonylamino, heterocycloalkyl, cycloalkyl and cycloalkylalkyl, any of which may be optionally substituted.

In further embodiments, the compounds of the present invention have structural Formula II wherein:

G¹ is selected from the group consisting of 2—N,N)-dimethylaminoethyl, 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, propyl, 2-chloro-4-bromophenyl, 2-fluoro-4-bromophenyl, and 2-fluoro-4-iodophenyl;

R¹ is selected from the group consisting of hydrogen, alkyl, alkoxyaminocarbonyl, and alkoxyaminocarbonylalkyl, any of which may be optionally substituted;

R³ is selected from the group consisting of the group consisting of hydrogen and alkyl, any of which may be optionally substituted;

X¹ is —N(R⁴)—;

R⁴ is selected from the group consisting of hydrogen, alkyl, cycloalkyl, and cycloalkylalkyl, any of which may be optionally substituted;

G² is selected from heteroaryl and a structure consisting of:

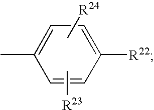

wherein:

R²² is selected from the group consisting of hydrogen, acyl, alkoxy, alkoxycarbonyl, alkyl, halogen, cyano, carboxylate, alkoxycarbonylalkyl, alkanoyl, alkylthio, cycloalkyl and cycloalkylalkyl, any of which may be optionally substituted; and

R²³ and R²⁴ are independently selected from the group consisting of hydrogen, acyl, alkoxy, alkoxycarbonyl, alkyl, halogen, cyano, carboxylate, perhaloalkyl, alkoxycarbonylalkyl, alkylaminocarbonyl, alkylaminocarbonylalkyl, alkanoyl, alkylthio, alkylsulfonyl, alkylsulfonylamino, heterocycloalkyl, cycloalkyl and cycloalkylalkyl, any of which may be optionally substituted.

In further embodiments, the compounds of the present invention have structural Formula H wherein:

G¹ is selected from the group consisting of 2-(N,N)-dimethylaminoethyl, 2-thienylmethyl, 4-fluorobenzyl, tetrahydro-2H-pyran4-yl, 4-hydroxycyclohexyl, 3-hydroxypropyl, 2-pyridylethyl, 3-pyridylethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-2-hydroxyethoxy)-carboxamido, and N-2,3-dihydroxypropoxy)-carboxamido;

R³ is hydrogen;

X¹ is —N(R⁴)—;

R⁴ is selected from the group consisting of hydrogen, alkyl, cycloalkyl, and cycloalkylalkyl, any of which may be optionally substituted;

G² is selected from heteroaryl and a structure consisting of:

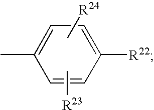

wherein:

R²² is selected from the group consisting of hydrogen, acyl, alkoxy, alkoxycarbonyl, alkyl, halogen, cyano, carboxylate, alkoxycarbonylalkyl, alkanoyl, alkylthio, cycloalkyl and cycloalkylalkyl, any of which may be optionally substituted; and

R²³ and R²⁴ are independently selected from the group consisting of hydrogen, acyl, alkoxy, alkoxycarbonyl, alkyl, halogen, cyano, carboxylate, perhaloalkyl, alkoxycarbonylalkyl, alkylaminocarbonyl, alkylaminocarbonylalkyl, alkanoyl, alkylthio, alkylsulfonyl, alkylsulfonylamino, heterocycloalkyl, cycloalkyl and cycloalkylalkyl, any of which may be optionally substituted.

In further embodiments, the compounds of the present invention have structural Formula H wherein:

G¹ is selected from the group consisting of 2—N,N)-dimethylaminoethyl, 2-thienylmethyl, 4-fluorobenzyl, tetrahydro-2H-pyran4-yl, 4-hydroxycyclohexyl, 3-hydroxypropyl, 2-pyridylethyl, 3-pyridylethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-(2,3-dihydroxypropoxy)-carboxamido;

R³ is hydrogen;

X¹ is —N(R⁴)—;

R⁴ is selected from the group consisting of hydrogen, alkyl, cycloalkyl, and cycloalkylalkyl, any of which may be optionally substituted; and

G² is optionally substituted heteroaryl.

In yet further embodiments, the compounds of the present invention have structural Formula II wherein:

G¹ is selected from the group consisting of 2-(N,N)-dimethylaminoethyl, 2-thienylmethyl, 4-fluorobenzyl, tetrahydro-2H-pyran4-yl, 4-hydroxycyclohexyl, 3-hydroxypropyl, 2-pyridylethyl, 3-pyridylethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-(2-hydroxyethoxy)-carboxamido, and N-2,3-dihydroxypropoxy)-carboxamido;

R³ is hydrogen;

X¹ is —N(R⁴)—;

R⁴ is selected from the group consisting of hydrogen, alkyl, cycloalkyl, and cycloalkylalkyl, any of which may be optionally substituted; and

G² is selected from the group consisting of 2-pyridyl, 3-pyridyl, 4-pyridyl, 5-methoxy-3-pyridyl, 5-methyl-3-pyridyl, 5-chloro-3-pyridyl, 5-methoxycarbonyl-3-pyridyl, 5-carboxy-3-pyridyl, 5-cyano-3-pyridyl, 5-acetyl-3-pyridyl, 5-ethylthio-3-pyridyl, 5-cyclopropyl-3-pyridyl, 5-cyclobutylmethyl-3-pyridyl, 4-methoxy-2-thienyl, 5-methyl-2-thienyl, 5-chloro-3-thienyl, 5-methoxycarbonyl-2-thienyl, 5-carboxy-2-thienyl, 5-cyano-2-thienyl, 5-acetyl-2-thienyl, 4-ethylthio-2-thienyl, 5-cyclopropyl-2-thienyl, 5-cyclobutylmethyl-2-thienyl, 1-methyl-1H-pyrazol-4-yl, 1-ethyl-4-methoxy-1H-pyrazol-5-yl, 1-benzyl-3-ethyl-1H-pyrazol-4-yl, 1 -phenyl-4-bromo-1H-pyrazol-5-yl, 1-(2-pyridyl)-3 -methoxycarbonyl-1H-pyrazol-4-yl, 1-t-butyl-3-carboxy-1H-pyrazol-4-yl, 1-cyclopentyl-3-cyano-1H-pyrazol-4-yl, 1-methyl-3-acetyl-1H-pyrazol4-yl, 1-methyl4-isopropylthio-1H-pyrazol-5-yl, 1-methyl-3-cyclopropyl-1H-pyrazol-4-yl, 1-propyl-3-cyclobutylmethyl-1H-pyrazol-4-yl, oxazol-5-yl, 4-methoxyoxazol-5-yl, 4-methyloxazol-5-yl, 5-chlorooxazol4-yl, 4-methoxycarbonyloxazol-5-yl, 4-carboxyoxazol-5-yl, 5-cyanooxazol-4-yl, 2-acetyloxazol-5-yl, 4-ethylthiooxazol-4-yl, 5-cyclopropyloxazol-4-yl, 4-cyclopentylmethyloxazol-5 -yl, 1H-imidazol-5-yl, 2-methoxy-1H-imidazol-5-yl, 4-methyl-1H-imidazol-5-yl, 5-chloro-1H-imidazol-5-yl, 4-ethoxycarbonyl-1H-imidazol-5-yl, 4-carboxy-1H-imidazol-5-yl, 4-cyano-1H-imidazol-5-yl, 2-acetyl-1H-imidazol-5-yl, 4-butylthio-1H-imidazol-5-yl, 5-cyclohexyl-1H-imidazol-4-yl, and cyclopentylethyl-1H-imidazol-5-yl, 1-ethyl-1H-imidazol-5-yl, 1-cyclobutyl-2-methoxy-1H-imidazol-5-yl, 1,4-dimethyl-1H-imidazol-5-yl, 1-propyl-5-chloro-1H-imidazol-5-yl, 1-methyl-4- ethoxycarbonyl-1H-imidazol-5-yl, 1-butyl-4-carboxy-1H-imidazol-5-yl, 1-isopropyl-4-cyano-1H-imidazol-5-yl, 1-methyl- 2-acetyl-1H-imidazol-5-yl, 1-ethyl-4-butylthio-1H-imidazol-5-yl, 1-methyl-5-cyclohexyl-1H-imidazol-4-yl, and 1-cyclopropyl-4-yclopentylethyl-1H-imidazol-5 -yl.

In yet further embodiments, the compounds of the present invention have structural Formula H wherein:

G¹ is selected from the group consisting of 2-(N,N)-dimethylaminoethyl, 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, propyl, 2-chloro-4-bromophenyl, 2-fluoro-4-bromophenyl, and 2-fluoro-4-iodophenyl;

R¹ is selected from the group consisting of hydrogen, methyl, ethyl, N-2-hydroxyethoxy)-carboxamido, and N-(2,3-dihydroxypropoxy)-carboxamido;

R³ is hydrogen;

X¹ is —N(R⁴)—;

R⁴ is selected from the group consisting of hydrogen, alkyl, cycloalkyl, and cycloalkylalkyl, any of which may be optionally substituted;

G² has the structure:.

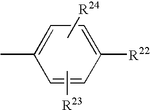

wherein:

R²² is selected from the group consisting of hydrogen, acetyl, methoxy, ethoxy, methoxycarbonyl, ethoxycarbonyl, methyl, ethyl, tert-butyl, fluoro, chloro, bromo, cyano, carboxylate, ethoxycarbonylmethyl, methylthio, isopropylthio, cyclopropyl, cyclopentyl, and cyclobutylethyl; and

R²³ and R²⁴ are independently selected from the group consisting of hydrogen, acetyl, methoxy, ethoxy, methoxycarbonyl, ethoxycarbonyl, methyl, ethyl, tert-butyl, fluoro, chloro, bromo, cyano, carboxylate, ethoxycarbonylmethyl, methylthio, isopropylthio, cyclopropyl, cyclopentyl, and cyclobutylethyl. methylaminocarbonyl, N,N′-dimethylaminocarbonyl, propanoyl, isobutanoyl, hexanoyl, methylsulfonyl, sec-butylsulfonyl, tetrahydropyran-4-yl, and ((tetrahydrofuran-2-yl)methyl).

In other embodiments, the compounds of the present invention have structural Formula III wherein:

R²⁵ is selected from the group consisting of hydrogen, acylamino, alkyl, alkoxycarbonyl, alkylamino, alkylaminocarbonyl, alkoxyaminocarbonyl, alkoxyaminocarbonylalkyl, alkylthio, alkylsulfonyl, amido, aminocarbonyl, arylalkoxy, aryl, arylalkyl, arylalkylamino, arylalkylthio, aryloxycarbonyl, arylamino, aryloxy, arylthio, cyano, cycloalkyl, cycloalkylalkyl, halogen, haloalkoxy, perhaloalkoxy, perhaloalkyl, heteroaryl, heteroarylalkyl, heteroaryloxy, heteroarylalkoxy, heterocycloalkyl, and nitro, any of which may be optionally substituted;

R²⁶ and R²⁸ are independently selected from the group consisting of hydrogen, acylamino, alkanoyl, alkyl, alkoxy, alkoxyalkyl, alkoxycarbonyl, alkoxycarbonylalkyl, alkylamino, kylaminocarbonyl, alkylaminocarbonylalkyl, alkoxyaminocarbonyl, alkoxyaminocarbonylalkyl, alkylcarbonyl, alkylthio, alkylsulfonyl, amido, aminocarbonyl, aminocarbonylalkyl, arylalkoxy, aryl, arylalkyl, arylalkylamino, arylalkylthio, aryloxycarbonyl, aralkanoyl, arylamino, aryloxy, arylthio, carboxy, cyano, cycloalkyl, cycloalkylalkyl, halogen, haloalkoxy, perhaloalkoxy, perhaloalkyl, heteroaryl, heteroarylalkyl, heteroaryloxy, heteroarylalkoxy, heterocycloalkyl, heterocycloalkylalkoxy, and nitro, any of which may be optionally substituted;

R²⁷ is selected from the group consisting of hydrogen, acylamino, alkanoyl, alkyl, alkoxy, alkoxyalkyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, arylalkoxy, aryl, arylalkyl, arylalkenyl, arylalkylamino, arylalkylthio, aryloxycarbonyl, aralkanoyl, arylamino, aryloxy, arylthio, carboxy, cyano, cycloalkyl, cycloalkylalkyl, halogen, haloalkoxy, perhaloalkoxy, perhaloalkyl, heteroaryl, heteroarylalkyl, heteroaryloxy, heteroarylalkoxy, heterocycloalkyl, heterocycloalkylalkoxy, and nitro, any of which may be optionally substituted;

R²⁹ is selected from the group consisting of acylamino, alkyl, alkenyl, alkynyl, alkoxyalkyl, alkoxycarbonyl, alkoxycarbonylalkyl, alkylaminocarbonyl, alkylaminocarbonylalkyl, arylalkyl, aryloxycarbonyl, aralkanoyl, carboxy, cyano, cycloalkyl, cycloalkylalkyl, perhaloalkyl, heteroaryl, heteroarylalkyl, and heterocycloalkyl, any of which may be optionally substituted;

Q¹⁰ is —N(R³⁰)—; and

R³⁰ is selected from the group consisting of aryl and heteroaryl, either of which may be optionally substituted.

In further embodiments, the compounds of the present invention have structural Formula III wherein:

R²⁵ is selected from the group consisting of hydrogen, alkyl, alkoxycarbonyl, alkylaminocarbonyl, alkoxyaminocarbonyl, alkylthio, alkylsulfonyl, aminocarbonyl, cyano, cycloalkyl, cycloalkylalkyl, halogen, perhaloalkoxy, and perhaloalkyl, any of which may be optionally substituted;

R²⁶ and R²⁸ are independently selected from the group consisting of hydrogen, alkyl, alkoxy, alkoxyalkyl, alkoxycarbonyl, alkylaminocarbonyl, alkoxyaminocarbonyl, alkylcarbonyl, alkylthio, alkylsulfonyl, cyano, cycloalkyl, cycloalkylalkyl, halogen, perhaloalkoxy, and perhaloalkyl, any of which may be optionally substituted;

R²⁷ is selected from the group consisting of hydrogen, acylamino, alkanoyl, alkyl, alkoxy, alkoxyalkyl, alkylaminocarbonyl, alkylaminocarbonylalkyl, alkoxyaminocarbonyl, alkylthio, alkylsulfonyl, cyano, cycloalkyl, cycloalkylalkyl, halogen, perhaloalkoxy, perhaloalkyl, and heterocycloalkyl, any of which may be optionally substituted;

R²⁹ is selected from the group consisting of alkyl, alkenyl, alkynyl, alkoxyalkyl, arylalkyl, cyano, cycloalkyl, cycloalkylalkyl, perhaloalkyl, heteroaryl, heteroarylalkyl, and heterocycloalkyl, any of which may be optionally substituted;

Q¹⁰ is —N(R³⁰)—; and

R³⁰ is optionally substituted aryl.

In further embodiments, the compounds of the present invention have structural Formula III wherein:

R²⁵ is selected from the group consisting of hydrogen, alkyl, alkoxycarbonyl, alkylaminocarbonyl, alkoxyaminocarbonyl, alkylthio, alkylsulfonyl, aminocarbonyl, cyano, cycloalkyl, cycloalkylalkyl, halogen, perhaloalkoxy, and perhaloalkyl, any of which may be optionally substituted;

R²⁶ and R²⁸ are independently selected from the group consisting of hydrogen, alkyl, alkoxy, alkoxyalkyl, alkoxycarbonyl, alkylaminocarbonyl, alkoxyaminocarbonyl, alkylcarbonyl, alkylthio, alkylsulfonyl, cyano, cycloalkyl, cycloalkylalkyl, halogen, perhaloalkoxy, and perhaloalkyl, any of which may be optionally substituted;

R²⁷ is selected from the group consisting of hydrogen, acylamino, alkanoyl, alkyl, alkoxy, alkoxyalkyl, alkylaminocarbonyl, alkylaminocarbonylalkyl, alkoxyaminocarbonyl, alkylthio, alkylsulfonyl, cyano, cycloalkyl, cycloalkylalkyl, halogen, perhaloalkoxy, perhaloalkyl, and heterocycloalkyl, any of which may be optionally substituted;

R²⁹ is selected from the group consisting of alkyl, alkenyl, alkynyl, alkoxyalkyl, arylalkyl, cyano, cycloalkyl, cycloalkylalkyl, perhaloalkyl, heteroaryl, heteroarylalkyl, and heterocycloalkyl, any of which may be optionally substituted;

Q¹⁰ is —N(R¹⁰)—; and

R³⁰ is selected from the group consisting of phenyl and phenyl optionally substituted with one or more of the substituents selected from the group consisting of acetylamino, cyclohexanoylamino, acetyl, propanoyl, methyl, ethyl, propyl, isopropyl, butyl, sec-butyl, isobutyl, tert-butyl, methoxy, ethoxy, butyloxy, isopropoxy, cyclohexylmethoxy, methoxyethyl, ethoxypropyl, benzyloxy, phenylethoxy, methoxycarbonyl, ethoxycarbonyl, methoxycarbonylmethyl, ethoxycarbonylmethyl, methylaminocarbonyl, ethylaminocarbonyl, diethylaminocarbonyl, ethylaminocarbonylmethyl, 3-hydroxy-1-propoxyaminocarbonyl, 2-methoxyethoxyaminocarbonyl, methythio, ethylthio, methylsulfonyl, butylsulfonyl, benzylsulfonyl, cyano, cyclopropyl, cyclobutyl, cyclohexyl, cyclopropylmethyl, cyclopropylethyl, cyclobutylmethyl, fluoro, chloro, bromo, difluoromethoxy, trifluoromethoxy, 2,2,2-trifluoroethoxy, and trifluoromethyl.

In yet fuirther embodiments, the compounds of the present invention have structural Formula III wherein:

R²⁵ and R²⁷ are hydrogen;

R²⁶ and R²⁸ are methoxy;

R²⁹ is methyl;

Q¹⁰ is —N(R³⁰)—; and

R ³⁰is phenyl.

In yet further embodiments, the compounds of the present invention have structural Formula III wherein:

R²⁵ is selected from the group consisting of hydrogen, alkyl, alkoxycarbonyl, alkylaminocarbonyl, alkoxyaminocarbonyl, alkylthio, alkylsulfonyl, aminocarbonyl, cyano, cycloalkyl, cycloalkylalkyl, halogen, perhaloalkoxy, and perhaloalkyl, any of which may be optionally substituted;

R²⁶ and R²⁸ are independently selected from the group consisting of hydrogen, alkyl, alkoxy, alkoxyalkyl, alkoxycarbonyl, alkylaminocarbonyl, alkoxyaminocarbonyl, alkylcarbonyl, alkylthio, alkylsulfonyl, cyano, cycloalkyl, cycloalkylalkyl, halogen, perhaloalkoxy, and perhaloalkyl, any of which may be optionally substituted;

R²⁷ is selected from the group consisting of hydrogen, acylamino, alkanoyl, alkyl, alkoxy, alkoxyalkyl, alkylaminocarbonyl, alkylaminocarbonylalkyl, alkoxyaminocarbonyl, alkylthio, alkylsulfonyl, cyano, cycloalkyl, cycloalkylalkyl, halogen, perhaloalkoxy, perhaloalkyl, and heterocycloalkyl, any of which may be optionally substituted;

R²⁹ is selected from the group consisting of alkyl, alkenyl, alkynyl, alkoxyalkyl, arylalkyl, cyano, cycloalkyl, cycloalkylalkyl, perhaloalkyl, heteroaryl, heteroarylalkyl, and heterocycloalkyl, any of which may be optionally substituted;

Q¹⁰ is —N(R³⁰)—; and

R³⁰ is optionally substituted heteroaryl.

In yet further embodiments, the compounds of the present invention have structural Formula III wherein:

R²⁵ is selected from the group consisting of hydrogen, alkyl, alkoxycarbonyl, alkylaminocarbonyl, alkoxyaminocarbonyl, alkylthio, alkylsulfonyl, aminocarbonyl, cyano, cycloalkyl, cycloalkylalkyl, halogen, perhaloalkoxy, and perhaloalkyl, any of which may be optionally substituted;

R²⁶ and R²⁸ are independently selected from the group consisting of hydrogen, alkyl, alkoxy, alkoxyalkyl, alkoxycarbonyl, alkylaminocarbonyl, alkoxyaminocarbonyl, alkylcarbonyl, alkylthio, alkylsulfonyl, cyano, cycloalkyl, cycloalkylalkyl, halogen, perhaloalkoxy, and perhaloalkyl, any of which may be optionally substituted;

R²⁷ is selected from the group consisting of hydrogen, acylamino, alkanoyl, alkyl, alkoxy, alkoxyalkyl, alkylaminocarbonyl, alkylaminocarbonylalkyl, alkoxyaminocarbonyl, alkylthio, alkylsulfonyl, cyano, cycloalkyl, cycloalkylalkyl, halogen, perhaloalkoxy, perhaloalkyl, and heterocycloalkyl, any of which may be optionally substituted;

R²⁹ is selected from the group consisting of alkyl, alkenyl, alkynyl, alkoxyalkyl, arylalkyl, cyano, cycloalkyl, cycloalkylalkyl, perhaloalkyl, heteroaryl, heteroarylalkyl, and heterocycloalkyl, any of which may be optionally substituted;

Q¹⁰ is —N(R³⁰)—; and

R³⁰ is selected from the group consisting of pyridyl, pyridazinyl, pyrimidinyl, pyrazinyl, 1,2,4-triazinyl, 1,3,5-triazinyl, thienyl, furyl, pyrrolyl, oxazolyl, isoxazolyl, thiazolyl, isothiazolyl, pyrazolyl, triazolyl, oxadiazolyl, and thiadiazolyl, any of which may be further optionally substituted with one or more of the substituents consisting of acetylamino, cyclohexanoylamino, acetyl, propanoyl, methyl, ethyl, propyl, isopropyl, butyl, sec-butyl, isobutyl, tert-butyl, methoxy, ethoxy, butyloxy, isopropoxy, cyclohexylmethoxy, methoxyethyl, ethoxypropyl, benzyloxy, phenylethoxy, methoxycarbonyl, ethoxycarbonyl, methoxycarbonylmethyl, ethoxycarbonylmethyl, methylaminocarbonyl, ethylaminocarbonyl, diethylaminocarbonyl, ethylaminocarbonylmethyl, 3-hydroxy-1-propoxyaminocarbonyl, 2-methoxyethoxyaminocarbonyl, methythio, ethylthio, methylsulfonyl, butylsulfonyl, benzylsulfonyl, cyano, cyclopropyl, cyclobutyl, cyclohexyl, cyclopropylmethyl, cyclopropylethyl, cyclobutylmethyl, fluoro, chloro, bromo, difluoromethoxy, trifluoromethoxy, 2,2,2-trifluoroethoxy, and trifluoromethyl.

The present invention further discloses a class of compounds, useful in treating MEK-mediated disorders and conditions, defined by structural Formula V:

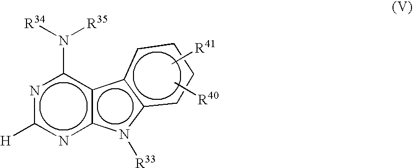

or a salt, ester, or prodrug thereof, wherein:

R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;

R³⁴ and R³⁵ are independently selected from the group consisting of hydrogen, acyl, alkyl, alkenyl, alkynyl, alkoxyalkyl, alkylaminoalkyl, alkylcarbonyl, amido, aminoalkyl, aryl, arylalkyl, cycloalkyl, cycloalkylalkyl, perhaloalkyl, heteroaryl, heteroarylalkyl, and heterocycloalkyl, any of which may be optionally substituted; or R³⁴and R³⁵, together with the atoms to which they are attached, may be joined to form an optionally substituted heterocycloalkyl moiety; and

R⁴⁰ and R⁴¹ are independently selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, carboxy, cyano, cycloalkyl, cycloalkylalkyl, halogen, haloalkyl, haloalkoxy, perhaloalkoxy, perhaloalkyl, hydroxy, hydroxyalkyl, nitro, and thiol, any of which may be optionally substituted.

In certain embodiments, the compounds of the present invention have structural Formula V wherein:

R³³ is selected from the group consisting of hydrogen, alkanoyl, alkyl, alkoxycarbonyl, alkylaminocarbonyl, alkylaminoalkyl, alkylsulfonyl, aminoalkyl, aminocarbonyl, cycloalkyl, and cycloalkylalkyl, any of which may be optionally substituted;

R³⁴ and R³⁵ are independently selected from the group consisting of hydrogen, alkyl, alkoxyalkyl, alkylaminoalkyl, aminoalkyl, cycloalkyl, cycloalkylalkyl, and heterocycloalkyl, any of which may be optionally substituted; or R³⁴ and R³⁵, together with the atoms to which they are attached, may be joined to form an optionally substituted heterocycloalkyl moiety; and

R⁴⁰ and R⁴¹ are independently selected from the group consisting of hydrogen, alkyl, alkoxy, alkoxycarbonyl, alkylamino, alkylaminocarbonyl, alkylaminoalkyl, alkylcarbonyl, alkylthio, alkylsulfonyl, amino, aminocarbonyl, carboxy, cyano, cycloalkyl, cycloalkylalkyl, halogen, perhaloalkyl, and nitro, any of which may be optionally substituted.

In further embodiments, the compounds of the present invention have structural Formula V wherein:

R³³ is selected from the group consisting of hydrogen, alkyl, alkylsulfonyl, cycloalkyl, and cycloalkylalkyl, any of which may be optionally substituted;

R³⁴ and R³⁵, together with the atoms to which they are attached, may be joined to form an optionally substituted heterocycloalkyl moiety; and

R⁴⁰ and R⁴¹ are independently selected from the group consisting of hydrogen, alkyl, alkoxy, alkylamino, alkylthio, alkylsulfonyl, cyano, cycloalkyl, cycloalkylalkyl, halogen, perhaloalkyl, and nitro, any of which may be optionally substituted.

In further embodiments, the compounds of the present invention have structural Formula V wherein:

R³³ is hydrogen;

R³⁴ and R³⁵ form morpholinyl; and

R⁴⁰ and R⁴¹ are hydrogen.

The present invention further discloses a class of compounds, useful in treating MEK-mediated disorders and conditions, defined by structural Formula VI:

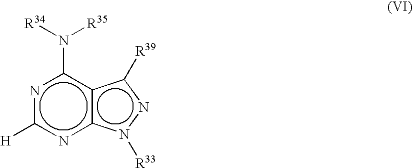

or a salt, ester, or prodrug thereof wherein:

R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;

R³⁴ and R³⁵ are independently selected from the group consisting of hydrogen, acyl, alkyl, alkenyl, alkynyl, alkoxyalkyl, alkylaminoalkyl, alkylcarbonyl, amido, aminoalkyl, aryl, arylalkyl, cycloalkyl, cycloalkylalkyl, perhaloalkyl, heteroaryl, heteroarylalkyl, and heterocycloalkyl, any of which may be optionally substituted; and R³⁴ and R³⁵, together with the atoms to which they are attached, may be joined to form an optionally substituted heterocycloalkyl moiety; and

R³⁹ is selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted.

In further embodiments, the compounds of the present invention have structural Formula VI wherein:

R³³ is selected from the group consisting of hydrogen, alkanoyl, alkyl, alkenyl, alkynyl, alkylsulfonyl, aminoalkyl, cycloalkyl, and cycloalkylalkyl, any of which may be optionally substituted;

R³⁴and R³⁵ are independently selected from the group consisting of hydrogen, alkyl, alkenyl, alkynyl, alkoxyalkyl, alkylaminoalkyl, aminoalkyl, cycloalkyl, and cycloalkylalkyl, any of which may be optionally substituted; and R³⁴and R³⁵, together with the atoms to which they are attached, may be joined to form an optionally substituted heterocycloalkyl moiety; and

R³⁹ is selected from the group consisting of hydrogen, alkyl, alkenyl, alkynyl, aryl, arylalkyl, cyano, cycloalkyl, cycloalkylalkyl, halogen, perhaloalkoxy, perhaloalkyl, heteroaryl, heteroarylalkyl, and heterocycloalkyl, any of which may be optionally substituted.

In further embodiments, the compounds of the present invention have structural Formula VI wherein:

R³³ is selected from the group consisting of hydrogen, alkyl, alkenyl, alkynyl, cycloalkyl, and cycloalkylalkyl, any of which may be optionally substituted;

R³⁴ and R³⁵ are independently selected from the group consisting of hydrogen, alkyl, alkoxyalkyl, alkylaminoalkyl, and aminoalkyl, any of which may be optionally substituted; and

R³⁹ is selected from the group consisting aryl and heteroaryl, either of which may be optionally substituted.

In yet further embodiments, the compounds of the present invention have structural Formula VI wherein:

R³³ is tert-butyl;

R³⁴ and R³⁵ are hydrogen; and

R³⁹ is 4-fluorophenyl.

The present invention fuirther discloses a class of compounds, useful in treating MEK-mediated disorders and conditions, defined by structural Formula VII:

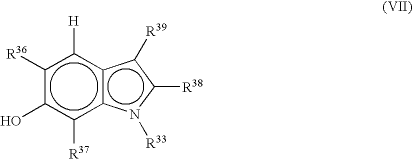

or a salt, ester, or prodrug thereof, wherein:

R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;

R³⁶ and R³⁷ are independently selected from the group consisting of hydrogen, alkyl, alkenyl, and alkynyl, any of which may be optionally substituted; and

R³⁸and R³⁹ are independently selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted.

In further embodiments, the compounds of the present invention have structural Formula VII wherein:

R³³ is selected from the group consisting of hydrogen, alkyl, alkenyl, alkynyl, alkoxycarbonyl, alkylsulfonyl, aryl, arylalkyl, aralkanoyl, cycloalkyl, cycloalkylalkyl, heteroaryl, heteroarylalkyl, and heterocycloalkyl, any of which may be optionally substituted;

R³⁶ and R³⁷ are independently selected from the group consisting of hydrogen, alkyl, alkenyl, and alkynyl, any of which may be optionally substituted; and

R³⁸and R³⁹ are independently selected from the group consisting of hydrogen, acylamino, alkanoyl, alkyl, alkoxy, alkoxycarbonyl, alkylaminocarbonyl, 1, alkylcarbonyl, alkylthio, alkylsulfonyl, amino, aminocarbonyl, aminocarbonylalkyl, carboxy, cyano, cycloalkyl, cycloalkylalkyl, halogen, haloalkoxy, perhaloalkoxy, perhaloalkyl, hydroxy, and nitro, any of which may be optionally substituted.

In further embodiments, the compounds of the present invention have structural Formula VII wherein:

R³³ is optionally substituted aryl;

R³⁶ and R³⁷ are selected from the group consisting of hydrogen and alkyl;

R³⁸ is selected from the group consisting of hydrogen, alkyl, cycloalkyl, and cycloalkylalkyl, any of which may be optionally substituted; and

R³⁹ are independently selected from the group consisting of alkoxy, alkoxycarbonyl, alkylsulfonyl, amino, aminocarbonyl, carboxy, cyano, halogen, perhaloalkoxy, perhaloalkyl, hydroxy, and nitro, any of which may be optionally substituted.

In yet further embodiments, the compounds of the present invention have structural Formula VII wherein:

R³³ is phenyl;

R³⁶ and R³⁷ are hydrogen;

R³⁸ is methyl; and

R³⁹ is nitro.

The present invention further discloses a class of compounds, useful in treating MEK-mediated disorders and conditions, defined by structural Formula VIII:

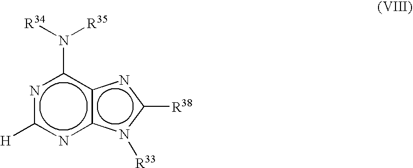

or a salt, ester, or prodrug thereof, wherein:

R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;

R³⁴ and R³⁵ are independently selected from the group consisting of hydrogen, acyl, alkyl, alkenyl, alkynyl, alkoxyalkyl, alkylaminoalkyl, alkylcarbonyl, amido, aminoalkyl, aryl, arylalkyl, cycloalkyl, cycloalkylalkyl, perhaloalkyl, heteroaryl, heteroarylalkyl, and heterocycloalkyl, any of which may be optionally substituted; or R³⁴and R³⁵, together with the atoms to which they are attached, may be joined to form an optionally substituted heterocycloalkyl moiety; and

R³⁸ is selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted.

In further embodiments, the compounds of the present invention have structural Formula VIII wherein:

R³³ is selected from the group consisting of hydrogen, alkanoyl, alkyl, alkoxyalkyl, alkoxycarbonyl, alkylaminocarbonyl, alkylaminoalkyl, alkylsulfonyl, amido, aminoalkyl, and aminocarbonyl, any of which may be optionally substituted;

R³⁴ and R³⁵ are independently selected from the group consisting of hydrogen, alkyl, alkenyl, alkynyl, alkoxyalkyl, alkylaminoalkyl, aminoalkyl, aryl, arylalkyl, cycloalkyl, cycloalkylalkyl, heteroaryl, heteroarylalkyl, and heterocycloalkyl, any of which may be optionally substituted; or R³⁴and R³⁵, together with the atoms to which they are attached, may be joined to form an optionally substituted heterocycloalkyl moiety; and

R³⁸ is selected from the group consisting of hydrogen, alkyl, alkenyl, alkynyl, cyano, cycloalkyl, and cycloalkylalkyl, any of which may be optionally substituted.

In further embodiments, the compounds of the present invention have structural Formula VIII wherein:

R³³ is selected from the group consisting of hydrogen, alkanoyl, alkoxyalkyl, alkoxycarbonyl, alkylsulfonyl, amido, and aminocarbonyl, any of which may be optionally substituted;

R³⁴ and R³⁵ are independently selected from the group consisting of hydrogen, alkyl, aryl, arylalkyl, cycloalkyl, cycloalkylalkyl, heteroaryl, heteroarylalkyl, and heterocycloalkyl, any of which may be optionally substituted; or R³⁴and R³⁵, together with the atoms to which they are attached, may be joined to form an optionally substituted heterocycloalkyl moiety; and

R³⁸ is selected from the group consisting of hydrogen and alkyl, either of which may be optionally substituted.

In yet further embodiments, the compounds of the present invention have structural Formula VIII wherein:

R³³ is selected from the group consisting of hydrogen, alkoxycarbonyl, and alkylsulfonyl, any of which may be optionally substituted;

R³⁴ and R³⁵ are independently selected from the group consisting of hydrogen, alkyl, aryl, arylalkyl, cycloalkyl, and heterocycloalkyl, any of which may be optionally substituted; or R³⁴and R³⁵, together with the atoms to which they are attached, may be joined to form an optionally substituted heterocycloalkyl moiety; and

R³⁸ is selected from the group consisting of hydrogen and alkyl, either of which may be optionally substituted.

In further embodiments, the compounds of the present invention have structural Formula VIII wherein:

R³³ is hydrogen;

R³⁴ is selected from the group consisting of hydrogen, cyclopentyl, and cycloheptyl;

R³⁵ is selected from the group consisting of hydrogen and ethyl; or R³⁴and R³⁵, together with the atoms to which they are attached, form 1-indolinyl and 4-methyl-1-piperidinyl; and

R³⁸ is hydrogen.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-diethylamino and the like.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (C:::C—, C≡—C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH- group, with R as defined herein.

The term “amino,” as used herein, alone or in combination, refers to —NRR, wherein R and R are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted as defined herein with one, two, three, four, or five substituents.

The term “aminoalkyl,” as used herein, alone or in combination, refers to an amino group attached to the parent molecular moiety through an alkyl group. Examples include aminomethyl, aminoethyl and aminobutyl.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl—O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄=derived from benzene. Examples include benzothiophene and benzimidazole.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′-group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O- group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.

The term “ester,” as used herein, alone or in combination, refers to a carboxyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2—NH—OCH3.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and I to 3 nitrogen atoms [e.g. benzoxazolyl, benzoxadiazolyl, etc.]; unsaturated 3 to 6-membered heteromonocyclic groups containing 1 to 2 sulfuir atoms and I to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.]and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylalkyl having five ring atoms” as used herein, alone or in combination, refers to a 5 membered unsaturated heterocyclic ring attached to an optionally substituted alkyl group, wherein at least one ring atom is selected from the group consisting of O, S, and N. Such groups are exemplified by: unsaturated 5 membered heteromonocyclic groups containing 1 to 4 nitrogen atoms, for example, pyrrolyl, pyrrolinyl, imidazolyl, pyrazolyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, unsaturated 3 to 6-membered heteromonocyclic groups containing an oxygen atom, for example, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and I to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; and unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.] and isothiazolyl.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfuir, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkylalkenyl,” as used herein, alone or in combination, refers to a heterocycle group attached to the parent molecular moiety through an alkenyl group.

The term “heterocycloalkylalkoxy,” as used herein, alone or in combination, refers to a heterocycle group attached to the parent molecular group through an oxygen atom.

The term “heterocycloalkylalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N— O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R is as defined herein.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NR′— group with R and R¹ as defined herein.

The term “S-sulfonamido” refers to a —S(═O)₂NR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NR, group with R as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term tri substituted silyl,“as used herein, alone or in combination, refers to a silicone group substituted at its three free valences with groups as listed herein under the definition of substituted amino. Examples include trimethysilyl, tert-butyldimethylsilyl, triphenylsilyl and the like.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“MEK inhibitor” is used herein to refer to a compound that exhibits an IC₅₀ with respect to MEK) activity of no more than about 100 μM and more typically not more than about 50 μM, as measured in the Mek1 kinase assay described generally hereinbelow. “IC₅₀” is that concentration of inhibitor which reduces the activity of an enzyme (e.g., MEK) to half-maximal level. Representative compounds of the present invention have been discovered to exhibit inhibition against MEK. Compounds of the present invention preferably exhibit an IC₅₀ with respect to MEK of no more than about 10 μM, more preferably, no more than about 5 μM, even more preferably not more than about 1 μM, and most preferably, not more than about 200 nM, as measured in the Mek1 kinase assay described herein.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines usefuil for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002).

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain anfioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include oral, intravenous, intraocular, subcutaneous, dermal, and inhaled topical.

For the treatment of oncologic diseases, proliferative disorders, and cancers, compounds according to the present invention may be administered with an agent selected from the group comprising: aromatase inhibitors, antiestrogen, anti-androgen, corticosteroids, gonadorelin agonists, topoisomerase 1and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, and aminopeptidase inhibitors.

For the treatment of oncologic diseases, proliferative disorders, and cancers, compounds according to the present invention may be administered with an agent selected from the group comprising: dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, carminomycin, idarubicin, epirubicin, esorubicin, mitoxantrone, bleomycins A, A₂, and B, camptothecin, Irinotecan®, Topotecan®, 9-aminocamptothecin, 10,11 -methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin®, Rituxan®, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva®, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

For the treatment of inflammatory diseases and pain, compounds according to the present invention may be administered with an agent selected from the group comprising: corticosteroids, non-steroidal anti-inflammatories, muscle relaxants and combinations thereof with other agents, anaesthetics and combinations thereof with other agents, expectorants and combinations thereof with other agents, antidepressants, anticonvulsants and combinations thereof; antihypertensives, opioids, topical cannabinoids, and other agents, such as capsaicin.

For the treatment of inflammatory diseases and pain, compounds according to the present invention may be administered with an agent selected from the group comprising: betamethasone dipropionate (augmented and nonaugemnted), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab; n) nitric oxide synthase inhibitors, particularly inhibitors of inducible nitric oxide synthase; and other agents, such as capsaicin.

For the treatment of ophthalmologic disorders and diseases of the eye, compounds according to the present invention may be administered with an agent selected from the group comprising: beta-blockers, carbonic anhydrase inhibitors, α- and β-adrenergic antagonists including al-adrenergic antagonists, α2 agonists, miotics, prostaglandin analogs, corticosteroids, and immunosuppressant agents.

For the treatment of ophthalmologic disorders and diseases of the eye, compounds according to the present invention may be administered with an agent selected from the group comprising: timolol, betaxolol, levobetaxolol, carteolol, levobunolol, propranolol, brinzolamide, dorzolamide, nipradilol, iopidine, brimonidine, pilocarpine, epinephrine, latanoprost, travoprost, bimatoprost, unoprostone, dexamethasone, prednisone, methylprednisolone, azathioprine, cyclosporine, and immunoglobulins.

For the treatment of autoimmune disorders, compounds according to the present invention may be administered with an agent selected from the group comprising: corticosteroids, immunosuppressants, prostaglandin analogs and antimetabolites.

For the treatment of autoimmune disorders, compounds according to the present invention may be administered with an agent selected from the group comprising: dexamethasome, prednisone, methylprednisolone, azathioprine, cyclosporine, immunoglobulins, latanoprost, travoprost, bimatoprost, unoprostone, infliximab, rutuximab and methotrexate.

For the treatment of metabolic disorders, compounds according to the present invention may be administered with an agent selected from the group comprising: insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, and non-glitazone type PPARS agonist.

For the treatment of metabolic disorders, compounds according to the present invention may be administered with an agent selected from the group comprising: insulin, metformin, Glipizide, glyburide, Amaryl, meglitinides, nateglinide, repaglinide, PT-112, SB-517955, SB4195052, SB-216763, NN-57-05441, NN-57-05445, GW-0791, AGN-¹⁹4²⁰4, T-1095, BAY R3401, acarbose Exendin-4, DPP728, LAF237, vildagliptin , MK-0431, saxagliptin, GSK23A, pioglitazone, rosiglitazone, (R)-1-{4-[5-methyl-2-(4-trifluoromethyl-phenyl)-oxazol-4-ylmethoxy]-benzenesulfonyl}2,3-dihydro-1H-indole-2-carboxylic acid described in the patent application WO 03/043985, as compound 19 of Example 4, and GI-262570.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating MEK kinase-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of MEK kinase-mediated disorders.

The invention also extends to the prophylaxis or treatment of any disease or disorder in which MEK kinase plays a role including, without limitation: oncologic, hematologic, inflammatory, ophthalmologic, neurological, immunologic, cardiovascular, and dermatologic diseases as well as diseases caused by excessive or unregulated pro-inflammatory cytokine production including for example excessive or unregulated TNF, IL-1, IL-6 and IL-8 production in a human, or other mammal. The invention extends to such a use and to the use of the compounds for the manufacture of a medicament for treating such cytokine-mediated diseases or disorders. Further, the invention extends to the administration to a human an effective amount of a MEK inhibitor for treating any such disease or disorder.

Diseases or disorders in which MEK kinase plays a role, either directly or via pro-inflammatory cytokines including the cytokines TNF, IL-1, IL-6 and IL-8, include, without limitation: dry eye, glaucoma, autoimmune diseases, inflammatory diseases, destructive-bone disorders, proliferative disorders, neurodegenerative disorders, viral diseases, allergies, infectious diseases, heart attacks, angiogenic disorders, reperfusion/ischemia in stroke, vascular hyperplasia, organ hypoxia, cardiac hypertrophy, thrombin-induced platelet aggregation, and conditions associated with prostaglandin endoperoxidase synthetase-2 (COX-2).

In certain aspects of the invention, the disease is a hyperproliferative condition of the human or animal body, including, but not limited to cancer, hyperplasias, restenosis, inflammation, immune disorders, cardiac hypertrophy, atherosclerosis, pain, migraine, angiogenesis-related conditions or disorders, proliferation induced after medical conditions, including but not limited to surgery, angioplasty, or other conditions.

In further embodiments, said hyperproliferative condition is selected from the group consisting of hematologic and nonhematologic cancers. In yet further embodiments, said hematologic cancer is selected from the group consisting of multiple myeloma, leukemias, and lymphomas. In yet fuirther embodiments, said leukemia is selected from the group consisting of acute and chronic leukemias. In yet further embodiments, said acute leukemia is selected from the group consisting of acute lymphocytic leukemia (ALL) and acute nonlymphocytic leukemia (ANLL). In yet further embodiments, said chronic leukemia is selected from the group consisting of chronic lymphocytic leukemia (CLL) and chronic myelogenous leukemia (CML). In further embodiments, said lymphoma is selected from the group consisting of Hodgkin's lymphoma and non-Hodgkin's lymphoma. In further embodiments, said hematologic cancer is multiple myeloma. In other embodiments, said hematologic cancer is of low, intermediate, or high grade. In other embodiments, said nonhematologic cancer is selected from the group consisting of: brain cancer, cancers of the head and neck, lung cancer, breast cancer, cancers of the reproductive system, cancers of the digestive system, pancreatic cancer, and cancers of the urinary system. In further embodiments, said cancer of the digestive system is a cancer of the upper digestive tract or colorectal cancer. In fuirther embodiments, said cancer of the urinary system is bladder cancer or renal cell carcinoma. In further embodiments, said cancer of the reproductive system is prostate cancer.

Additional types of cancers which may be treated using the compounds and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancer s may beselected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compounds and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

In some aspects of the invention, the disease to be treated by the methods of the present invention may be a hematologic disorder. In certain embodiments, said hematologic disorder is selected from the group consisting of sickle cell anemia, myelodysplastic disorders (MDS), and myeloproliferative disorders. In further embodiments, said myeloproliferative disorder is selected from the group consisting of polycythemia vera, myelofibrosis and essential thrombocythemia.

The compositions of the present invention are useful as anti-inflammatory agents with the additional benefit of having significantly less harmful side effects. The compositions are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compositions are also useful in treating osteoporosis and other related bone disorders. These compositions can also be used to treat gastrointestinal conditions such as reflux esophagitis, diarrhea, inflammatory bowel disease, Crohn's disease, gastritis, irritable bowel syndrome and ulcerative colitis. The compositions may also be used in the treatment of pulmonary inflammation, such as that associated with viral infections and cystic fibrosis. In addition, compositions of invention are also useful in organ transplant patients either alone or in combination with conventional immunomodulators. Yet further, the compositions of the invention are useful in the treatment of pruritis and vitaligo. The invention further extends to the particular inflammatory disease rheumatoid arthritis.

Further inflammatory diseases which may be prevented or treated include, without limitation: asthma, allergies, respiratory distress syndrome or acute or chronic pancreatitis. Furthermore, respiratory system diseases may be prevented or treated including but not limited to chronic obstructive pulmonary disease, and pulmonary fibrosis. In addition, MEK kinase inhibitors of this invention are also associated with prostaglandin endoperoxidase synthetase-2 (COX-2) production. Pro-inflammatory mediators of the cyclooxygenase pathway derived from arachidonic acid, such as prostaglandins, are produced by inducible COX-2 enzyme. Regulation of COX-2 would regulate these pro-inflammatory mediators, which affect a wide variety of cells and are important and critical inflammatory mediators of a wide variety of disease states and conditions. In particular, these inflammatory mediators have been implicated in pain, such as in the sensitization of pain receptors, and edema. Accordingly, additional MEK kinase-mediated conditions which may be prevented or treated include edema, analgesia, fever and pain such as neuromuscular pain, headache, dental pain, arthritis pain and pain caused by cancer.

In some aspects of the invention, the disease to be treated by the methods of the present invention may be an ophthalmologic disorder. Ophthalmologic diseases and other diseases in which angiogenesis plays a role in pathogenesis, may be treated or prevented and include, without limitation, dry eye (including Sjogren's syndrome), macular degeneration, closed and wide angle glaucoma, retinal ganglion degeneration, occular ischemia, retinitis, retinopathies, uveitis, ocular photophobia, and of inflammation and pain associated with acute injury to the eye tissue. The compositions can be used to treat glaucomatous retinopathy and/or diabetic retinopathy. The compositions can also be used to treat post-operative inflammation or pain as from ophthalmic surgery such as cataract surgery and refractive surgery.. In further embodiments, said ophthalmologic disorder is selected from the group consisting of dry eye, closed angle glaucoma and wide angle glaucoma.

In some aspects of the invention, the disease to be treated by the methods of the present invention may be an autoimmune disease. Autoimmune diseases which may be prevented or treated include, but are not limited to: rheumatoid arthritis, inflammatory bowel disease, inflammatory pain, ulcerative colitis, Crohn's disease, periodontal disease, temporomandibular joint disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs. host disease, and psoriasis. Inflammatory diseases which may be prevented or treated include, but are not limited to: asthma, allergies, respiratory distress syndrome or acute or chronic pancreatitis. The invention further extends to the particular autoimmune disease rheumatoid arthritis.

In some aspects of the invention, the disease to be treated by the methods of the present invention may be a dermatologic disorder. In certain embodiments, said dermatologic disorder is selected from the group including, without limitation, melanoma, basel cell carcinoma, squamous cell carcinoma, and other non-epithelial skin cancer as well as psoriasis and persistent itch, and other diseases related to skin and skin structure, may be treated or prevented with MEK kinase inhibitors of this invention.

Metabolic diseases which may be treated or prevented include, without limitation, metabolic syndrome, insulin resistance, and Type 1 and Type 2 diabetes. In addition, the compositions of the subject invention can be used to treat insulin resistance and other metabolic disorders such as atherosclerosis that are typically associated with an exaggerated inflammatory signaling.

The compositions of the present invention are also useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like. These compositions can also be used to treat allergic rhinitis, respiratory distress syndrome, endotoxin shock syndrome, and atherosclerosis.

In some aspects of the invention, the disease to be treated by the methods of the present invention may be a cardiovascular condition. In certain embodiments, said cardiovascular condition is selected from the group consisting of atherosclerosis, cardiac hypertrophy, idiopathic cardiomyopathies, heart failure, angiogenesis-related conditions or disorders, and proliferation induced after medical conditions, including, but not limited to restenosis resulting from surgery and angioplasty.

In other aspects of the invention, the disease to be treated by the methods of the present invention may be a neurological disorder. In certain embodiments, said neurologic disorder is selected from the group consisting of Parkinson's disease, Alzheimer's disease, Alzheimer's dementia, and central nervous system damage resulting from stroke, ischemia and trauma. In other embodiments, said neurological disorder is selected from the group consisting of epilepsy, neuropathic pain, depression and bipolar disorders.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

The following schemes can be used to practice the present invention. Starting materials are commercially available, made by known procedures, or prepared as illustrated herein.

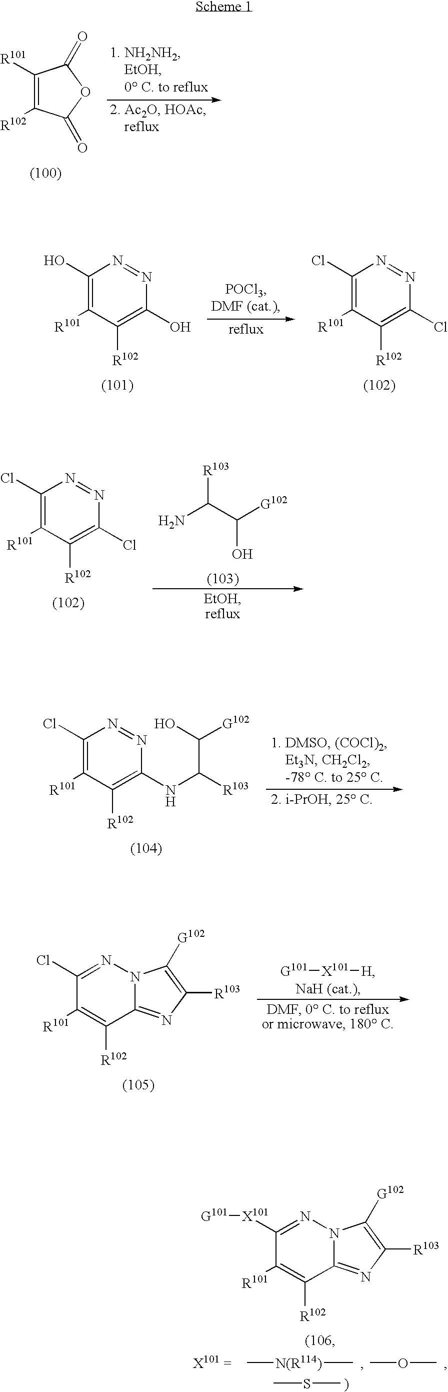

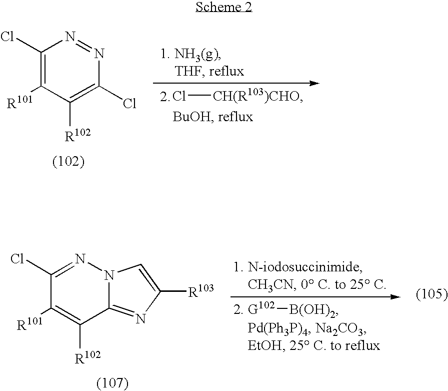

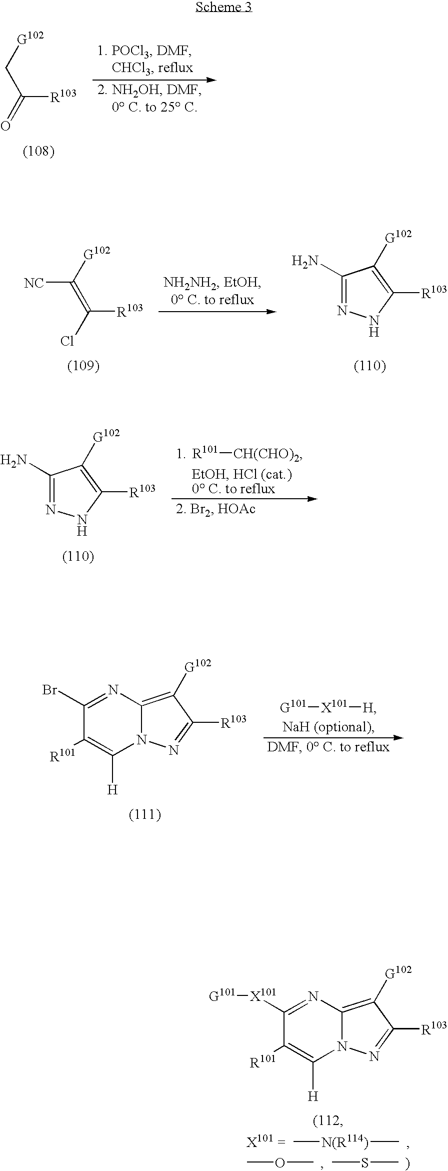

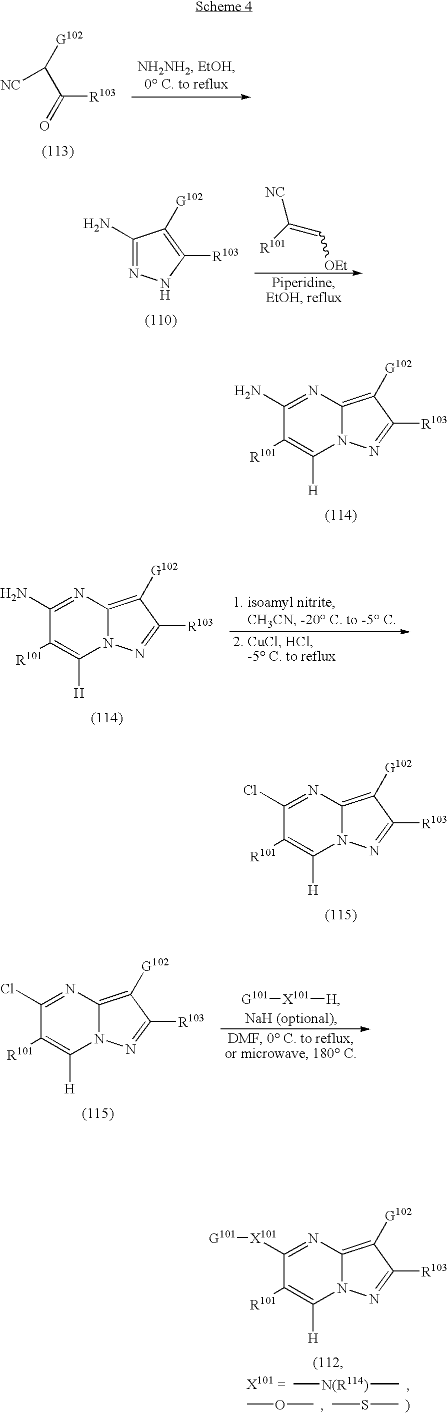

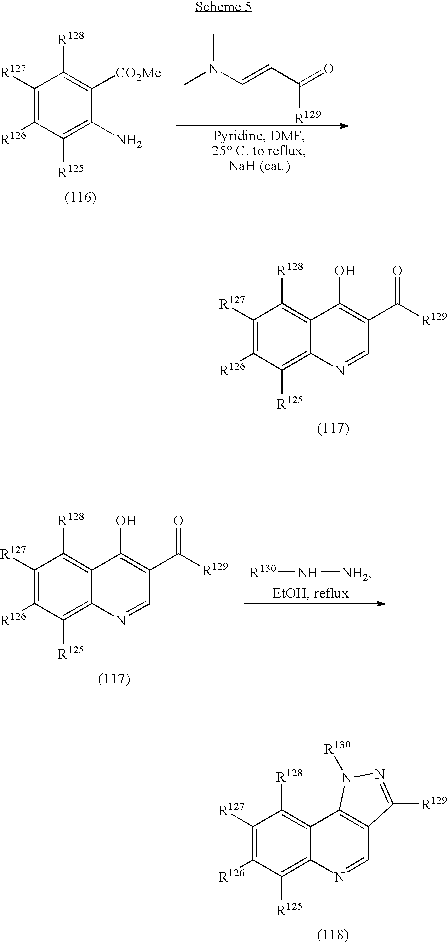

The following compounds (Examples 1 to 22, shown in Table 1 below) were synthesized by procedures similar to those as described above using the appropriate starting materials and by methods known in the art.

TABLE 1

| | | MS | |
|---|---|---|---|
| EX | STRUCTURE | (m/z) | NAME |
| 1 | 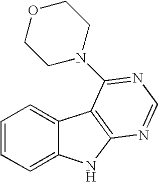 | 254.12 | 4-(9H-pyrimido[4,5-b]indol-4- yl)morpholine |
| 2 | 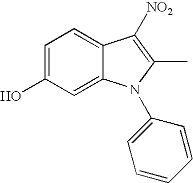 | 268.08 | 2-methyl-3-nitro-1-phenyl-1H- indol-6-ol |
| 3 | 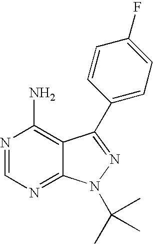 | 285.14 | 1-tert-butyl-3-(4- fluorophenyl)-1H- pyrazolo[3,4-d]pyrimidin-4- amine |
| 4 | 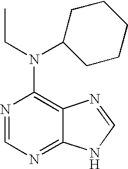 | 245.16 | N-cyclohexyl-N-ethyl-9H- purin-6-anime |
| 5 | 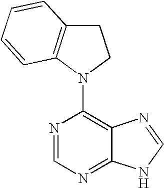 | 237.10 | 6-(indolin-1-yl)-9H-purine |
| 6 | 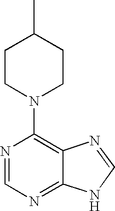 | 217.13 | 6-(4-methylpiperidin-1-yl)-9H- purine |
| 7 | 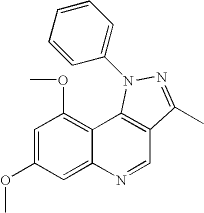 | 319.00 | 7,9-dimethoxy-3-methyl-1- phenyl-1H-pyrazolo[4,3- c]quinoline |
| 8 | 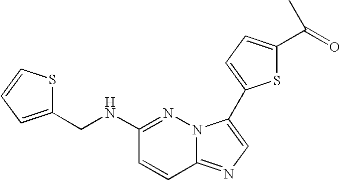 | 354.00 | 1-(5-(6-(thiophen-2- ylmethylamino)imidazo[1,2- b]pyridazin-3-yl)thiophen-2- yl)ethanone |
| 9 | 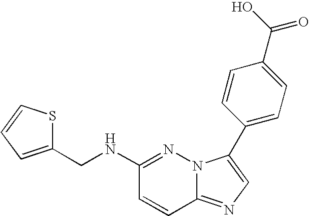 | 350.00 | 4-(6-(thiophen-2- ylmethylamino)imidazo[1,2- b]pyridazin-3-yl)benzoic acid |
| 10 | 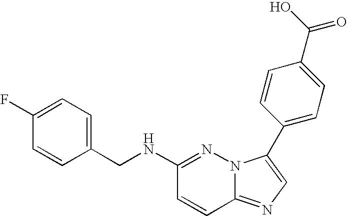 | 362.00 | 4-(6-(4- fluorobenzylamino)imidazo[1, 2-b]pyridazin-3-yl)benzoic acid |
| 11 | 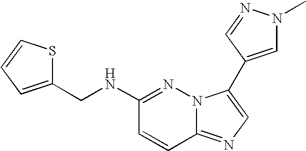 | 310.00 | 1-methyl-1H-pyrazol-4-yl)- N-(thiophen-2- ylmethyl)imidazo[1,2- b]pyridazin-6-amine |
| 12 | 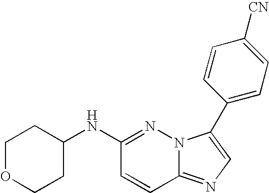 | 319.00 | 4-(6-(tetrahydro-2H-pyran-4- ylamino)imidazo[1,2- b]pyridazin-3-yl)benzonitrile |
| 13 | 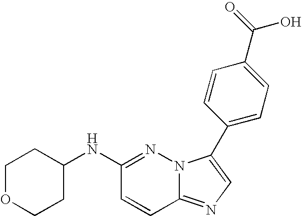 | 338.00 | 4-(6-(tetrahydro-2H-pyran-4- ylamino)imidazo[1,2- b]pyridazin-3-yl)benzoic acid |
| 14 | 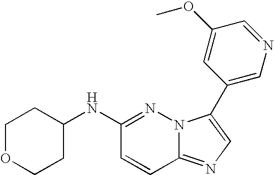 | 325.00 | 3-(5-methoxypyridin-3-yl)-N- (tetrahydro-2H-pyran-4- yl)imidazo[1,2-b]pyridazin-6- amine |
| 15 | 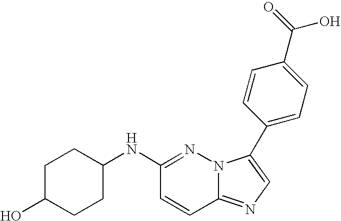 | 352.00 | 4-(6-(4- hydroxycyclohexylamino) imidazo[1,2-b]pyridazin-3- yl)benzoic acid |
| 16 | 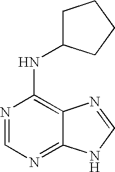 | 231.15 | N-cyclopentyl-9H-purin-6- amine |
| 17 | 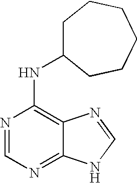 | 203.00 | N-cycloheptyl-9H-purin-6- amine |
| 18 | 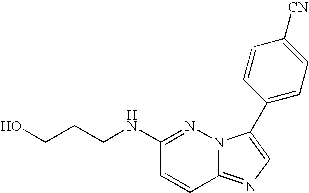 | 293.13 | 4-(6-(3- hydroxypropylamino)imidazo [1,2-b]pyridazin-3- yl)benzonitrile |
| 19 | 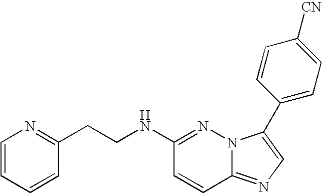 | 340.14 | 4-(6-(2-(pyridin-2- yl)ethylamino)imidazo[1,2- b]pyridazin-3-yl)benzonitrile |
| 20 | 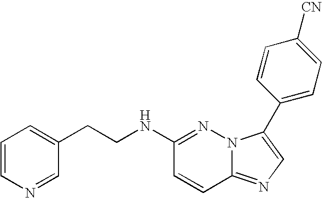 | 340.14 | 4-(6-(2-(pyridin-3- yl)ethylamino)imidazo[1,2- b]pyridazin-3-yl)benzonitrile |
| 21 | 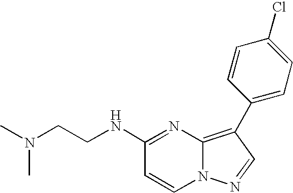 | 315.00 | N1-(3-(4- chlorophenyl)pyrazolo[1,5- a]pyrimidin-5-yl)-N2,N2- dimethylethane-1,2-diamine |
| 22 | 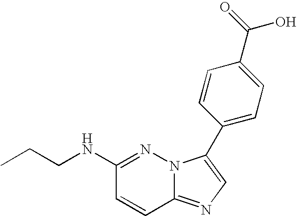 | 296.00 | 4-(6- (propylamino)imidazo[1,2- b]pyridazin-3-yl)benzoic acid |

The following compounds can generally be made using the both literature methods and those described above. It is expected that these compounds when made will have activity similar to those that have been made in the examples above. The compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf Comput. Sci. 1988, 28, 31-36.

BrC(C═C1)═CC(C1)═C1NC(C(C(NOCCO)═O)═C2)═NN3C2═NC═C3C4═CC═CC═C4 BrC(C═C1)═CC(F)═C1NC(C(C(NOCCO)═O)═C2)═NN3C2═NC═C3C4═CC═CC═C4 IC(C═C1)═CC(F)═C1NC(C(C(NOCCO)═O)═C2)═NN3C2═NC═C3C4═CC═CC═C4 BrC(C═C1)═CC(C1)═C1NC(C(C(NOCC(O)CO)═O)═C2)═NN3C2═NC═C3C4═CC═CC═C4 BrC(C═C1)═CC(F)═C1NC(C(C(NOCC(O)CO)═O)═C2)═NN3C2═NC═C3C4═CC═CC═C4 IC(C═C1)═CC(F)═C1NC(C(C(NOCC(O)CO)═O)═C2)═NN3C2═NC═C3C4═CC═CC═C4 BrC(C═C1)═CC(C1)═C1NC(C(C(NOCCO)═O)═C2)═NN3C2═NC═C3C4═CC═CN═C4 BRC(C═C1)═CC(F)═C1NC(C(C(NOCCO)═O)═C2)═NN3C2═NC═C3C4═CC═CN═C4 IC(C═C1)═CC(F)═C1NC(C(C(NOCCO)═O)═C2)═NN3C2═NC═C3C4═CC═CN═C4 BRC(C═C1)═CC(C1)═C1NC(C(C(NOCC(O)CO)═O)═C2)═NN3C2═NC═C3C4═CC═CN═C4 BRC(C═C1)═CC(F)═C1NC(C(C(NOCC(O)CO)═O)═C2)═NN3C2═NC═C3C4═CC═CN═C4 IC(C═C1)═CC(F)═C1NC(C(C(NOCC(O)CO)═O)═C2)═NN3C2═NC═C3C4═CC═CN═C4

BrC(C═C1)═CC(C1)═C1NC2═NC3═C(C4═CC═CC═C4)C═NN3C═C2C(NOC CO)═O BrC(C═C1)═CC(F)═C1NC2═NC3═C(C4═CC═CC═C4)C═NN3C═C2C(NOCCO)═O IC(C═C1)═CC(F)═C1NC2═NC3═C(C4═CC═CC═C4)C═NN3C═C2C(NOCCO)═O BrC(C═C1)═CC(C1)═C1NC2═NC3═C(C4═CC═CC═C4)C═NN3C═C2C(NOCC(O) CO)═O BrC(C═C1)═CC(F)═C1NC2═NC3═C(C4═CC═CC═C4)C═NN3C═C2C(NOCC(O)CO)═O IC(C═C1)═CC(F)═C1NC2═NC3═C(C4═CC═CC═C4)C═NN3C═C2C(NOCC(O)CO)═O BrC(C═C1)═CC(C1)═C1NC2═NC3═C(C4═CC═CN═C4)C═NN3C═C2C(NOC CO)═O BrC(C═C1)═CC(F)═C1NC2═NC3═C(C4═CC═CN═C4)C═NN3C═C2C(NOCCO)═O IC(C═C1)═CC(F)═C1NC2═NC3═C(C4═CC═CN═C4)C═NN3C═C2C(NOCCO)═O BrC(C═C1)═CC(C1)═C1NC2═NC3═C(C4═CC═CN═C4)C═NN3C═C2C(NOC C(O)CO)═O BrC(C═C1)═CC(F)═C1NC2═NC3═C(C4═CC═CN═C4)C═NN3C═C2C(NOCC(O)CO)═O IC(C═C1)═CC(F)═C1NC2═NC3═C(C4═CC═CN═C4)C═NN3C═C2C(NOCC(O)CO)═O BrC(C═C1)═CC(C1)═C1NC2═NC3═C(C4═CN═CO4)C═NN3C═C2C(NOCCO)═O BrC(C═C1)═CC(F)═C1NC2═NC3═C(C4═CN═CO4)C═NN3C═C2C(NOCCO)═O IC(C═C1)═CC(F)═C1NC2═NC3═C(C4═CN═CO4)C═NN3C═C2C(NOCCO)═O BrC(C═C1)═CC(C1)═C1NC2═NC3═C(C4═CN═CO4)C═NN3C═C2C(NOCC(O)C O)═O BrC(C═C1)═CC(F)═C1NC2═NC3═C(C4═CN═CO4)C═NN3C═C2C(NOCC(O)CO)═O IC(C═C1)═CC(F)═C1NC2═NC3═C(C4═CN═CO4)C═NN3C═C2C(NOCC(O)CO)═O BrC(C═C1)═CC(C1)═C1NC2═NC3═C(C4═CN═CN4)C═NN3C═C2C(NOCCO)═O BrC(C═C1)═CC(F)═C1NC2═NC3═C(C4═CN═CN4)C═NN3C═C2C(NOCCO)═O IC(C═C1)═CC(F)═C1NC2═NC3═C(C4═CN═CN4)C═NN3 C═C2C(NOCCO)═O BrC(C═C1 )═CC(C1)═C1NC2═NC3═C(C4═CN═CN4)C═NN3C═C2C(NOCC(O)C O)═O BrC(C═C1)═CC(F)═C1NC2═NC3═C(C4═CN═CN4)C═NN3C═C2C(NOCC(O)CO)═O IC(C═C1)═CC(F)═C1NC2═NC3═C(C4═CN═CN4)C═NN3C═C2C(NOCC(O)CO)═O

The compounds in Examples I through 22 have been shown to be KSP inhibitors by using the following assays. The other compounds listed above, which have not yet been made and/or tested, are predicted to have activity in these assays as well.

### In vitro Mek1 Kinase Assay

2.5 μl of Mek1 kinase buffer (10 mM MOPS [pH 7.2], 25 mM sodium glycerophosphate, 5 mM EGTA [pH 8.0], 1 mM sodium orthovanadate, 1 mM dithiothreitol, 10 mM MgCl₂, 0.1% Brij-35, 0.3 mg/ml bovine serum albumin) containing either 5 ng of high specific activity, recombinant N-terminal GST-tagged, C-terminal His6-tagged human phospho-Mek1 (Upstate Cat. 14-429), or 200 ng of low specific activity, non-phosphorylated Mek1 (Upstate Cat. 14-420), is dispensed into one well of a 1536 multi-well white solid plate. 50 nl of 100× concentration of test compound in dimethyl sulfoxide (DMSO) is dispensed to the well by passive pin transfer and incubated for 15 minutes at room temperature (22° C.). 2.5 μl of Mek1 kinase buffer containing 2 μM ATP is then dispensed and the kinase reaction is allowed to incubate at 30° C. for 2 hours. The assay plates are lidded and maintained in a humidified environment. After 2 hours, 2.5 μl of PKLight protein kinase assay reagent (Cambrex) is dispensed. After an additional 5 minute incubation at room temperature, luminescence activity is measured on a suitable multi-mode plate reader. Inhibition of Mek1 kinase causes an associated increase in luminescence activity that is proportional to compound concentration. Negative control activity is measured with DMSO lacking any test compound. Positive control activity is measured with (R)—N— (2,3-dihydroxypropoxy)-3,4-fluoro4-iodophenylamino)benzamide [Thompson, N. and Lyons, J., Curr Opin Pharmacol, 5:350-356, 2005 (PD-0325901)]. Efficacy is measured as a percentage of positive control activity. Results are shown below in Table 2.

TABLE 2

| | IC₅₀: (−): ≦10 μM; |
|---|---|
| Example# | (+): >10 μM |
| 1 | (−) |
| 2 | (−) |
| 3 | (−) |
| 4 | (−) |
| 5 | (+) |
| 6 | (+) |
| 7 | (−) |
| 8 | (+) |
| 9 | (−) |
| 10 | (−) |
| 11 | (−) |
| 12 | (−) |
| 13 | (−) |
| 14 | (+) |
| 15 | (−) |
| 16 | (+) |
| 17 | (+) |
| 18 | (−) |
| 19 | (−) |
| 20 | (−) |
| 21 | (+) |
| 22 | (+) |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I: 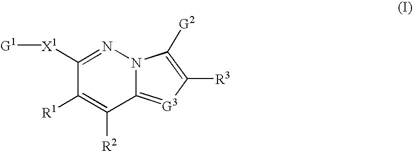
or a salt, ester, or prodrug thereof, wherein:
G¹ is selected from the group consisting of alkyl, aminoalkyl, hydroxyalkyl, haloalkyl, perhaloalkyl, acyl, alkenyl, alkynyl, heteroalkyl, cycloalkyl, cycloalkylalkyl, heterocycloalkyl, aryl, arylalkyl, arylalkenyl, arylalkynyl, heteroaryl, heteroarylalkyl, heteroarylalkenyl, and heteroarylalkynyl, any of which may be optionally substituted;
R¹ and R² are independently selected from the group consisting of hydrogen, acylamino, alkanoyl, alkyl, alkenyl, alkynyl, alkoxy, alkoxyalkyl, alkoxycarbonyl, alkoxycarbonylalkyl, alkylamino, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;
R³ is selected from the group consisting of hydrogen, alkanoyl, alkyl, alkenyl, alkynyl, alkoxy, alkoxyalkyl, alkoxycarbonyl, alkoxycarbonylalkyl, alkylaminocarbonyl, alkylaminocarbonylalkyl, alkoxyaminocarbonyl, alkoxyaminocarbonylalkyl, alkylaminoalkyl, alkylcarbonyl, alkylthio, alkylsulfonyl, amido, aminoalkyl, aminocarbonyl, aminocarbonylalkyl, arylalkoxy, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted;
X¹ is selected from the group consisting of a bond, —N(R⁴)—, —O—, —S—, —SO—, —SO₂—, alkylene, alkenylene, perhaloalkylene, —C(═O)C(═O)—, —C(═O)O—, —C(═O)N(R⁵)—,—N(R⁵)C(═O)N(R⁶)—, —N(R⁵)SO₂N(R⁶)—, —N(R⁵)C(═O)O—, —C(═O)[C (R⁷)(R⁸)]ₘ—, —C(═O)O[C(R⁷)(R⁸)]ₘ—, —SO₂N(R⁵)—, —C(═S)—, —C(═O)S—, —S(═O)ₙ[C(R⁷)(R⁸)]ₘ, —P(═O)(OR⁹)—, —P(═O)(NR⁹)—, —P(═S)(OR⁹)—, —P(═S)(NR⁹)—, —S(═O)(═NR₉)—, and —S(═NR⁹)(═NR⁹)—, any of which may be optionally substituted;
R⁴, R⁵, R⁶, R⁷, R⁸, and R⁹are independently selected from the group consisting of hydrogen, acyl, alkoxycarbonyl, alkoxycarbonylalkyl, alkyl, alkylsulfonyl, alkenyl, alkynyl, alkoxyalkyl, alkylaminoalkyl, alkylcarbonyl, amido, aminoalkyl, aryl, arylalkyl, arylalkenyl, arylalkynyl, cycloalkyl, cycloalkylalkyl, haloalkyl, perhaloalkyl, heteroaryl, heteroarylalkyl, heteroarylalkenyl, and heterocycloalkyl, any of which may be optionally substituted; or R⁵ and R⁶, together with the atoms to which they are attached, may be joined to form an optionally substituted cycloalkyl or optionally substituted heterocycloalkyl moiety; or R⁷ and R⁸, together with the atoms to which they are attached, may be joined to form an optionally substituted cycloalkyl or optionally substituted heterocycloalkyl moiety;
G² is selected from a structure from the group consisting of: 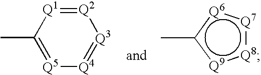
wherein:
Q¹ and Q⁵ are independently selected from the group consisting of —C(R¹⁰)—, and —N—;
Q² and Q⁴ are independently selected from the group consisting of —C(R¹¹)—, and —N—;
Q³ is selected from the group consisting of —C(R¹²)— and —N—;
Q⁶ is selected from the group consisting of —C(R¹³)—, —N—, —N(R¹⁴)—, —O—, and —S—;
Q⁷ is selected from the group consisting of —C(R¹⁵)—, —N—, —N(R¹⁶)—, —O—, and —S—;
Q⁸ is selected from the group consisting of —C(R¹³)—, —N—, —N(R¹⁶)—, —O—, and —S—;
Q⁹ is selected from the group consisting of —C(R¹⁷)—, —N—, —N(R¹⁸)—, —O—, and —S—;
R¹⁰, R¹³, R¹⁴, R¹⁷ and R¹⁸ are independently selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl and cycloalkylalkyl, any of which may be optionally substituted;
R¹¹ is selected from the group consisting of hydrogen, fluoro, bromo, iodo, cyano, nitro, hydroxy, acylamino, alkyl, alkylamino, alkoxy, alkoxyalkoxy, haloalkyl, perhaloalkyl, haloalkoxy, aryl, aralkyl, arylalkenyl, arylalkynyl, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylalkylamino, heteroaryloxy, heteroarylalkoxy, arylthio, arylsulfonyl, heteroarylthio, heteroarylalkylthio, heterocycloalkyl, cycloalkyl, and cycloalkylalkyl, any of which may be optionally substituted;
R¹² is selected from the group consisting of hydrogen, fluoro, bromo, iodo, cyano, nitro, hydroxy, acyl, acylamino, alkyl, alkylamino, alkoxy, alkoxyalkoxy, haloalkyl, perhaloalkyl, haloalkoxy, aryl, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, heteroarylalkylthio, heterocycloalkyl, cycloalkyl, and cycloalkylalkyl, any of which may be optionally substituted;
R¹⁵ is selected from the group consisting of hydrogen, halogen, cyano, nitro, hydroxy, acyl, acylamino, alkyl, alkylamino, haloalkyl, perhaloalkyl, aryl, aralkyl, arylalkenyl, arylalkynyl, arylamino, arylalkylamino, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroaryl, heteroarylalkyl, heteroarylalkenyl, heteroarylalkynyl, heteroarylamino, heteroarylalkylamino, arylthio, arylsulfonyl, heteroarylthio, heteroarylalkylthio, heterocycloalkyl, cycloalkyl, and cycloalkylalkyl, any of which may be optionally substituted;
R¹⁶ is selected from the group consisting of hydrogen, acyl, alkyl, perhaloalkyl, haloalkoxy, aralkyl, arylalkenyl, arylalkynyl, arylsulfonyl, alkenyl, alkynyl, alkoxycarbonyl, alkoxycarbonylalkyl, alkylaminocarbonyl, alkylaminocarbonylalkyl, alkanoyl, hydroxyalkyl, alkylsulfonyl, heteroarylalkyl, heteroarylalkenyl, heteroarylalkynyl, arylsulfonyl, heterocycloalkyl, cycloalkyl, and cycloalkylalkyl, any of which may be optionally substituted;
G³ is selected from the group consisting of —N— and C(R¹⁹);
R¹⁹ is selected from the group consisting of hydrogen, halogen, cyano, nitro, hydroxy, acyl, acylamino, alkyl, alkylamino, alkoxy, alkoxyalkoxy, haloalkyl, perhaloalkyl, haloalkoxy, aralkyl, arylalkenyl, arylalkynyl, arylamino, arylalkylamino, aryloxy, arylalkoxy, arylthio, arylsulfonyl, alkenyl, alkynyl, alkoxycarbonyl, alkoxycarbonylalkyl, alkylaminocarbonyl, alkylaminocarbonylalkyl, carboxylate, alkanoyl, hydroxyalkyl, alkylthio, alkylsulfonyl, alkylsulfonylamino, heteroarylalkyl, heteroarylalkenyl, heteroarylalkynyl, heteroarylamino, heteroarylalkylamino, heteroaryloxy, heteroarylalkoxy, arylthio, arylsulfonyl, heteroarylthio, and heteroarylalkylthio, any of which may be optionally substituted;
m is an integer from 1 to 5;
n is an integer from 0 to 2;
and provided that when X¹ is —NH—, and G¹ is 2-thienylmethyl, then G² may not be phenyl.

2. The compound as recited in claim 1, wherein:
G¹ is selected from the group consisting of alkyl, aminoalkyl, hydroxyalkyl, cycloalkyl, cycloalkylalkyl, heterocycloalkyl, aryl, arylalkyl, heteroaryl, and heteroarylalkyl, any of which may be optionally substituted;
R¹ and R²are independently selected from the group consisting of hydrogen, alkyl, alkoxy, alkylaminocarbonyl, alkylaminocarbonylalkyl, alkoxyaminocarbonyl, alkoxyaminocarbonylalkyl, alkylcarbonyl, alkylthio, amido, aminocarbonyl, aminocarbonylalkyl, cyano, cycloalkyl, cycloalkylalkyl, halogen, and perhaloalkyl, any of which may be optionally substituted;
R³ is selected from the group consisting of from the group consisting of hydrogen, alkyl, alkoxy, alkylthio, alkylsulfonyl, cyano, cycloalkyl, cycloalkylalkyl, halogen, and perhaloalkyl, any of which may be optionally substituted;
X¹ is selected from the group consisting of —N(R⁴)—, —O—, —S—, —SO₂—, —C(═O)N(R₅)—, —N(R⁵)C(═O)O—, and —SO₂N(R⁵)—, any of which may be optionally substituted;
R⁴ and R⁵ are independently selected from the group consisting of hydrogen, acyl, alkoxycarbonyl, alkoxycarbonylalkyl, alkyl, alkylsulfonyl, alkylcarbonyl, arylalkyl, heteroarylalkyl, cycloalkyl, and cycloalkylalkyl, any of which may be optionally substituted;
R¹⁰, R¹³, R¹⁴, R¹⁷, and R¹⁸ are independently selected from the group consisting of hydrogen, acyl, alkoxy, alkoxycarbonyl, alkyl, halogen, cyano, carboxylate, acylamino, alkyl, alkylamino, perhaloalkyl, alkylthio, alkylsulfonyl, alkylsulfonylamino, heteroarylalkyl, cycloalkyl, and cycloalkylalkyl, any of which may be optionally substituted;
R¹¹ is selected from the group consisting of hydrogen, fluoro, bromo, cyano, acylamino, alkyl, alkoxy, perhaloalkyl, alkoxycarbonyl, alkoxycarbonylalkyl, alkylaminocarbonyl, alkylaminocarbonylalkyl, alkylthio, alkylsulfonyl, alkylsulfonylamino, carboxylate, cycloalkyl, and cycloalkylalkyl, any of which may be optionally substituted;
R¹² is selected from the group consisting of hydrogen, fluoro, bromo, cyano, acylamino, alkyl, alkylamino, alkoxy, perhaloalkyl, arylamino, arylalkylamino, aryloxy, arylalkoxy, arylthio, arylsulfonyl, alkoxycarbonyl, alkylaminocarbonyl, alkylthio, alkylsulfonyl, alkylsulfonylamino, heteroarylamino, heteroarylalkylamino, heteroaryloxy, heteroarylalkoxy, arylthio, arylsulfonyl, heteroarylthio, heteroarylalkylthio, carboxy, cycloalkyl, and cycloalkylalkyl, any of which may be optionally substituted;
R¹⁵ is selected from the group consisting of hydrogen, acyl, alkoxycarbonyl, alkyl, halogen, cyano, carboxylate, perhaloalkyl, alkoxycarbonylalkyl, alkylaminocarbonyl, alkylaminocarbonylalkyl, alkylthio, alkylsulfonyl, alkylsulfonylamino, cycloalkyl, and cycloalkylalkyl, any of which may be optionally substituted;
R¹⁶ is selected from the group consisting of hydrogen, acyl, alkoxycarbonyl, alkyl, alkoxycarbonylalkyl, alkylaminocarbonyl, alkylaminocarbonylalkyl, alkylsulfonyl, arylsulfonyl, cycloalkyl, and cycloalkylalkyl, any of which may be optionally substituted; and
G³ is —N—.

3. The compound as recited in claim 2, wherein:
R¹ and R² are independently selected from the group consisting of hydrogen, alkyl, alkylaminocarbonyl, alkylaminocarbonylalkyl, alkoxyaminocarbonyl, alkoxyaminocarbonylalkyl, aminocarbonyl, aminocarbonylalkyl, cyano, cycloalkyl, cycloalkylalkyl, and halogen, any of which may be optionally substituted;
R³is selected from the group consisting of hydrogen, alkyl, cycloalkyl, cycloalkylalkyl, and perhaloalkyl, any of which may be optionally substituted;
X¹ is selected from the group consisting of —N(R⁴)—, —O—, and —S—, any of which may be optionally substituted;
R⁴ is selected from the group consisting of hydrogen, alkyl, cycloalkyl, and cycloalkylalkyl, any of which may be optionally substituted;
R¹⁰, R¹³, R¹⁴, R¹⁷, and R¹⁸ are independently selected from the group consisting of hydrogen, acyl, alkoxy, alkoxycarbonyl, alkyl, halogen, cyano, carboxylate, alkylthio, cycloalkyl, and cycloalkylalkyl, any of which may be optionally substituted;
R¹¹ is selected from the group consisting of hydrogen, fluoro, bromo, cyano, alkyl, alkoxy, alkoxycarbonyl, carboxylate, alkylthio, cycloalkyl, and cycloalkylalkyl, any of which may be optionally substituted; and
R¹² is selected from the group consisting of hydrogen, fluoro, bromo, cyano, alkyl, alkoxy, alkoxycarbonyl, alkylthio, carboxy, cycloalkyl, and cycloalkylalkyl, any of which may be optionally substituted.

4. The compound as recited in claim 3, wherein:
G¹ is selected from the group consisting of 2-thienylmethyl, 4-fluorobenzyl, tetrahydro-2H-pyran-4-yl, 4-hydroxycyclohexyl, 3-hydroxypropyl, 2-pyridylethyl, 3-pyridylethyl, 2-(N,N)-dimethylaminoethyl, propyl, 2-chloro-4-bromophenyl, 2-fluoro-4-bromophenyl, and 2-fluoro-4-iodophenyl;
R¹ is selected from the group consisting of hydrogen, methyl, ethyl, N-(2-hydroxyethoxy)-carboxamido, and N-(2,3-dihydroxypropoxy)-carboxamido;
R², R³, and R⁴ are hydrogen;
X¹ is —N(R⁴)—;
G² is selected from a structure consisting of: 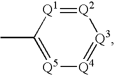
Q¹ and Q⁵ are —C(R¹⁰)—;
Q² and Q⁴ are —C(R¹¹)—;
Q³ is —C(R¹²)—;
R¹⁰ and R¹¹ are hydrogen;
R¹² is selected from the group consisting of hydrogen, carboxy and cyano; and
G³ is —N—.

5. The compound as recited in claim 4, wherein:
Q⁴ is —N—;
R¹⁰ and R¹² are hydrogen; and
R¹¹ is methoxy.

6. The compound as recited in claim 3, wherein:
G¹ is selected from the group consisting of 2-thienylmethyl, 4-fluorobenzyl, tetrahydro-2H pyran-4-yl, 4-hydroxycyclohexyl, 3-hydroxypropyl, 2-pyridylethyl, 3-pyridylethyl, 2—N,N)-dimethylaminoethyl, propyl, 2-chloro-4-bromophenyl, 2-fluoro-4-bromophenyl, and 2-fluoro-4-iodophenyl;
R¹ is selected from the group consisting of hydrogen, methyl, ethyl, N-(2-hydroxyethoxy)-carboxamido, and N-(2,3-dihydroxypropoxy)-carboxamido;
R², R³, and R⁴ are hydrogen;
X¹ is —N(R⁴)—;
G²_is selected from a structure consisting of: 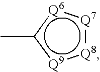
Q⁷ is —C(R¹⁵)—;
Q⁸ is —C(R¹³)—;
Q⁹ is —C(R¹⁷)—;
R¹⁵ is acetyl; and
R¹³ and R¹⁷ are hydrogen.

7. The compound as recited in claim 6, wherein:
Q⁶ is —C(R¹³)—;
Q⁷ is —N—;
Q¹ is —N(R⁶)—;
Q⁹ is —C(R¹⁷)—;
R¹³ and R¹⁷ are hydrogen; and
R¹⁶ is methyl.

8. A method of inhibiting MEK kinase in a patient in need thereof comprising the administration of a compound of structural Formula IV: 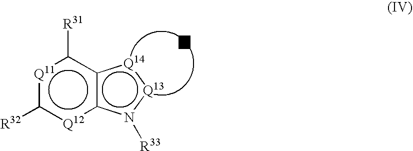
or a salt, ester, or prodrug thereof, wherein:
A is a carbocyclic, heterocyclic, aromatic, or heteroaromatic group, any of which may be optionally substituted and each of which have five to eight ring atoms including Q¹³ and Q¹⁴; or, alternatively, A may be absent;
R³¹ is selected from the group consisting of hydrogen and —N(R³⁴)(R³⁵);
R³² is selected from the group consisting of hydrogen and hydroxy;
R³³ is selected from the group consisting of hydrogen, alkanoyl, alkyl, alkenyl, alkynyl, alkoxyalkyl, alkoxycarbonyl, alkoxycarbonylalkyl, alkylaminocarbonyl, alkylaminocarbonylalkyl, alkylaminoalkyl, alkylsulfonyl, amido, aminoalkyl, aminocarbonyl, aminocarbonylalkyl, aryl, arylalkyl, aryloxycarbonyl, aralkanoyl, cycloalkyl, cycloalkylalkyl, heteroaryl, heteroarylalkyl, and heterocycloalkyl, any of which may be optionally substituted;
R³⁴ and R³⁵ are independently selected from the group consisting of hydrogen, acyl, alkyl, alkenyl, alkynyl, alkoxyalkyl, alkylaminoalkyl, alkylcarbonyl, amido, aminoalkyl, aryl, arylalkyl, cycloalkyl, cycloalkylalkyl, perhaloalkyl, heteroaryl, heteroarylalkyl, and heterocycloalkyl, any of which may be optionally substituted; and R³⁴ and R³⁵, together with the atoms to which they are attached, may be joined to form an optionally substituted heterocycloalkyl moiety;
Q¹¹ is selected from the group consisting of —N— and —C(R³⁶)—;
Q¹² is selected from the group consisting of —N— and —C(R³⁷)—;
Q¹³ is selected from the group consisting of —N— and —C(R³⁸)—;
Q¹⁴ is selected from the group consisting of —N— and —C(R³⁹)—;
R³⁶ and R³⁷ are independently selected from the group consisting of hydrogen, alkyl, alkenyl, and alkynyl, any of which may be optionally substituted; and
R³⁹ and R³⁹ are independently selected from the group consisting of hydrogen, acylamino, alkanoyl, alkyl, alkenyl, alkynyl, alkoxy, alkoxycarbonyl, alkylamino, alkylaminocarbonyl, alkylaminocarbonylalkyl, alkylaminoalkyl, alkylcarbonyl, alkylthio, alkylsulfonyl, amido, amino, aminoalkyl, aminocarbonyl, aminocarbonylalkyl, arylalkoxy, aryl, arylalkyl, arylalkenyl, arylalkylamino, arylalkylthio, arylalkynyl, aryloxycarbonyl, aralkanoyl, arylamino, aryloxy, arylthio, carboxy, cyano, cycloalkyl, cycloalkylalkyl, halogen, haloalkyl, haloalkoxy, perhaloalkoxy, perhaloalkyl, heteroaryl, heteroarylalkyl, heteroarylalkenyl, heteroaryloxy, heteroarylalkoxy, heterocycloalkyl, heterocycloalkylalkoxy, hydroxy, hydroxyalkyl, nitro, and thiol, any of which may be optionally substituted.

9. The method as recited in claim 8 comprising the administration of a compound of structural Formula V: 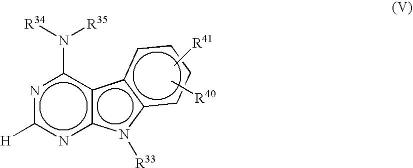
or a salt, ester, or prodrug thereof, wherein:
R³³ is hydrogen;
R³⁴ and R³⁵ are morpholinyl; and
R⁴⁰ and R⁴¹ are hydrogen.

10. The method as recited in claim 8 comprising the administration of a compound of structural Formula VI: 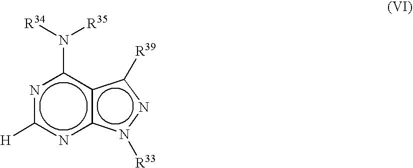
or a salt, ester, or prodrug thereof, wherein:
R³³ is tert-butyl;
R³⁴ and R³⁵ are hydrogen; and
R³⁹ is 4-fluorophenyl.

11. The method as recited in claim 8 comprising the administration of a compound of structural Formula VH: 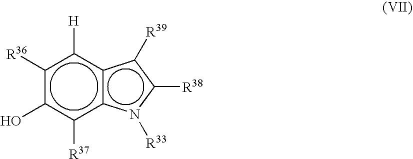
or a salt, ester, or prodrug thereof, wherein:
R³³ is phenyl;
R³⁶ and R³⁷ are hydrogen;
R³⁸ is methyl; and
R³⁹ is nitro.

12. The method as recited in claim 8 comprising the administration of a compound of structural Formula VIII: 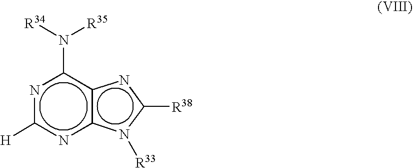
or a salt, ester, or prodrug thereof, wherein:
R³³ is hydrogen;
R³⁴ is selected from the group consisting of hydrogen, cyclopentyl, and cycloheptyl;
R³⁵ is selected from the group consisting of hydrogen and ethyl; and R³⁴ and R³⁵, together with the atoms to which they are attached, form 1-indolinyl and 4-methyl-1-piperidinyl; and
R³⁸ is hydrogen.

13. A compound as recited in claim 1 for use as a medicament.

14. A compound as recited in claim 1 for use in the manufacture of a medicament for the prevention or treatment of a disease or condition ameliorated by the inhibition of MEK kinase.

15. A pharmaceutical composition comprising a compound as recited in claim 1 together with a pharmaceutically acceptable carrier.

16. A pharmaceutical composition comprising at least one compound selected from the group consisting of those recited in Example 1 and Examples 3 to 22, together with a pharmaceutically acceptable carrier.

17. A method of inhibiting MEK kinase comprising contacting MEK kinase with a compound as recited in claim 1.

18. A method of inhibiting MEK kinase comprising contacting MEK kinase with a compound as recited in claim 8.

19. A method of treatment of a MEK kinase-mediated disease comprising the administration of a therapeutically effective amount of a compound as recited in claim 1 to a patient in need thereof.

20. The method as recited in claim 19 wherein said disease is a hyperproliferative disease.

21. A method of treatment of a MEK kinase-mediated disease comprising the administration of a therapeutically effective amount of a compound as recited in claim 8 to a patient in need thereof.

22. The method as recited in claim 21 wherein said disease is a hyperproliferative disease.

23. A method of treatment of a MEK kinase-mediated disease comprising the administration of:
a. a therapeutically effective amount of a compound as recited in claim 1; and
b. another therapeutic agent.

24. The method as recited in claim 23 wherein said diseases are cancers, non-cancer hyperproliferative disorders, vascular restenosis, psoriasis, autoimmune disorders, atherosclerosis, rheumatoid arthritis, osteoarthritis, heart failure, chronic pain, neuropathic pain, dry eye, closed angle glaucoma and wide angle glaucoma.

25. A method of treatment of a MEK kinase-mediated disease comprising the administration of:
a. a therapeutically effective amount of a compound as recited in claim 8; and
b. another therapeutic agent.

26. A method for achieving an effect in a patient comprising the administration of a therapeutically effective amount of a compound as recited in claim 1 to a patient, wherein the effect is selected from the group consisting of inhibition of various cancers, immunological diseases, and inflammatory diseases.

27. A method for achieving an effect in a patient comprising the administration of a therapeutically effective amount of a compound as recited in claim 8 to a patient, wherein the effect is selected from the group consisting of inhibition of various cancers, immunological diseases, and inflammatory diseases.
